(19) United States
(12) Patent Application Publication
(10) Pub. No.: US 2024/0336677 A1
(43) Pub. Date: Oct. 10, 2024

(54) COMPOSITIONS AND METHODS FOR TREATING ALPHA-SYNUCLEINOPATHIES

(71) Applicants: The Regents of the University of Michigan, Ann Arbor, MI (US); The Johns Hopkins University, Baltimore, MD (US)

(72) Inventors: Wenjing Wang, Ann Arbor, MI (US); Xiaobo Mao, Baltimore, MD (US); Yemima Rianik, Ann Arbor, MI (US); Yuqing Liu, Baltimore, MD (US); Ramhari Kumbhar, Baltimore, MD (US)

(21) Appl. No.: 18/579,227

(22) PCT Filed: Jul. 14, 2022
(86) PCT No.: PCT/US22/37038

§ 371 Date: Jan. 12, 2024

Related U.S. Application Data
(60) Provisional application No. 63/222,141, filed on Jul. 15, 2021.

(51) Int. Cl. C07K 16/18; A61K 45/06; A61K 48/00; A61P 25/28

(52) U.S. Cl. CPC C07K 16/18; A61K 45/06; A61K 48/0033; A61K 48/0075; A61P 25/28; C07K 2317/569

(57) ABSTRACT

Provided herein are compositions and methods to prevent and to treat α-synucleinopathies for research and therapeutic purposes. In particular, provided herein are compositions, methods, kits and uses for inhibition of pathological phosphorylation and spread of α-synuclein in the central nervous system as therapeutic targets of neurodegenerative disorders including dementia with Lewy bodies and Parkinson's disease dementia.

### CROSS-REFERENCE TO RELATED APPLICATIONS

The present application claims priority to U.S. Provisional Application Ser. No. 63/222,141 filed Jul. 15, 2021, the entirety of which is incorporated by reference herein.

This invention was made with government support under AG072009 awarded by the National Institutes of Health. The government has certain rights in the invention.

### FIELD

Provided herein are compositions and methods to prevent and to treat α-synucleinopathies for research and therapeutic purposes. In particular, provided herein are compositions, methods, kits and uses for inhibition of pathological phosphorylation and spread of α-synuclein in the central nervous system as therapeutic targets of neurodegenerative disorders including dementia with Lewy bodies and Parkinson's disease dementia.

### BACKGROUND

Lewy body dementia (LBD) is one of the most common causes of dementia, including Parkinson's disease with dementia (PDD) and dementia with Lewy bodies (DLB)¹⁻³. Approximately 30% of Alzheimer' disease (AD) patients also suffer from LBD resulting in a more rapid and severe cognition decline than AD alone^{4,5}. LBD belongs to α-synuclein disorders also termed α-synucleinopathies, which are associated with abnormal deposits of a protein called α-synuclein (α-syn) aggregates in the cortex^{6,7}. Post-mortem studies on PDD patients by Braak et al. show that misfolded α-synuclein (“α-syn”) is a prion-like protein, and its pathology spreads in a stereotypical fashion⁸⁻¹⁰. A single administration of recombinant α-syn preformed fibrils (PFF) seeds aggregation of endogenous α-syn monomer, and subsequently induces α-syn spreading pathology¹¹⁻¹⁵. Both clinical and experimental observations support prion-like α-syn driving α-synucleinopathy expression.

Antibodies have been developed against α-syn for research and therapeutic purposes. Antibodies against α-syn oligomer or protofibrillar form may be used to determine the maturation stages in PD pathology by immunostaining of the pathological tissues¹⁶⁻¹⁹. A human-derived α-syn antibody, BIIB054, shows high selectivity against the aggregated forms over the monomer form of α-syn. BIIB054 prevents the spread of α-syn pathology in mouse models and it is in a Phase 2 clinical trial²⁰. Despite their promise, long-term administration of antibodies is costly and inconvenient. Intracellular expression of single chain antibody fragment (scFv) D5 that binds to the oligomeric form of α-syn inhibits α-syn pathology.^{21,22} However, intracellular expression of scFV aggregates thereby compromising its therapeutic effects²².

Nanobodies provide an alternative to scFV as an intrabody and offer multiple advantages. Nanobodies, also termed “single-domain antibodies”, are small in size (for example, about 15 kDa)²³. They are more stable than conventional antibodies and scFV in the reducing environment of the cell because nanobodies contain only one disulfide bond that participates in their folding. Due to their small size and stability, nanobodies may be modified to cross blood-brain barrier²⁴, and may be expressed intracellularly in different cell lines²⁵. Several nanobodies against α-syn have been described, including NbSyn2 and NbSyn87^{26,27}. NbSyn87 also interacts with the proteasome and reduces PD pathology both in vitro and in vivo^{28,29}. However, neither NbSyn2 nor NbSyn87 distinguish α-syn monomer from PFF, and threaten to perturb the function of physiological α-syn in healthy cells. Therefore, there is a need for nanobodies that preferentially bind to α-syn PFF but not the α-syn monomer to minimize functional perturbations of the α-syn monomer in healthy cells. Nanobodies contain a disulfide bond that stabilizes the folding under oxidizing conditions. However, the disulfide bond is reduced in the cytosol giving rise to differences in nanobody folding and stability. Thus, it is advantageous to develop disulfide bond-free nanobodies to ensure consistent folding under both reducing and oxidizing conditions for intracellular applications. Accordingly, there is a need for nanobodies that preferentially bind to toxic α-syn PFF but not the α-syn monomer to minimize functional perturbations of the monomer in healthy cells of use in compositions and methods for preventing and treating Lewy Body Dementia and related disorders.

### SUMMARY

Provided herein are compositions and methods to prevent and to treat α-synucleinopathies for research and therapeutic purposes. In particular, provided herein are compositions, methods, kits and uses for inhibition of pathological phosphorylation and spread of α-synuclein in the central nervous system as therapeutic targets of neurodegenerative disorders including dementia with Lewy bodies and Parkinson's disease dementia.

Provided herein are methods of treating an α-synucleinopathy, comprising exposing cells of a subject's central nervous system (CNS) to an α-synuclein preformed fibrils (PFF) inhibitor wherein said exposing treats said α-synucleinopathy. In some embodiments, the subject is a human subject. In certain embodiments, the α-synucleinopathy is Lewy body dementia (LBD), Parkinson's disease with dementia (PDD) and/or dementia with Lewy bodies (DLB). In given embodiments, the α-synuclein preformed fibrils (PFF) inhibitor is an anti-α-synuclein preformed fibrils (PFF) antibody. In particular embodiments, the anti-α-synuclein preformed fibrils (PFF) antibody is an anti-α-synuclein preformed fibrils (PFF) nanobody. In specific embodiments, the anti-α-synuclein preformed fibrils (PFF) nanobody does not comprise a disulfide bond. In some embodiments, the anti-α-synuclein preformed fibrils (PFF) nanobody is fibril-specific. In certain embodiments, the anti-α-synuclein preformed fibrils (PFF) nanobody is encoded into an adeno-associated virus (AAV) vector. In given embodiments, the anti-α-synuclein preformed fibrils (PFF) nanobody is encoded by PFFNB2 (SEQ ID NO. 1) and/or PFFNB7 (SEQ ID NO. 2). In specific embodiments, the AAV vector is administered by intracerebroventricular administration.

In given embodiments, the α-synuclein preformed fibrils (PFF) inhibitor interferes with α-synuclein preformed fibrils (PFF) expression. In further embodiments, the exposing is in vivo exposing, ex vivo exposing and/or in vitro exposing. In still further embodiments, the exposing to said α-synuclein preformed fibrils (PFF) inhibitor is selected from the group consisting of local administration, topical administration, intrathecal administration, intraparenchymal administration, intracerebroventrical administration, intravenous administration, intraarterial administration, intrapulmonary administration, and oral administration. In some embodiments the exposing comprises combination therapy with an agent that treats α-synucleinopathy. In specific embodiments, the agent is selected from the group consisting of a small molecule, a peptide, and a nucleic acid.

Provided herein are methods of treating an α-synucleinopathy disease in a subject, comprising assaying a plasma and/or cerebrospinal fluid (CSF) sample from a subject, and administering an agent that is an α-synuclein preformed fibrils (PFF) inhibitor.

Provided herein are compositions comprising an α-synuclein preformed fibrils (PFF) inhibitor and a pharmaceutically acceptable carrier. In some embodiments the anti-α-synuclein preformed fibrils (PFF) antibody is an anti-α-synuclein preformed fibrils (PFF) nanobody. In certain embodiments, the anti-α-synuclein preformed fibrils (PFF) nanobody is encoded into an adeno-associated virus (AAV) vector. In specific embodiments, the anti-α-synuclein preformed fibrils (PFF) nanobody is encoded by PFFNB2 (SEQ ID NO. 1) and/or PFFNB7 (SEQ ID NO. 2).

In some embodiments, provided herein are methods, compositions, kits and systems for the generation of disulfide-free nanobody libraries to identify nanobodies against additional targets of interest, for example, targets for which conventional monoclonal antibodies are poor candidates, and/or targets for which intracellular stability of a nanobody is preferred.

In some embodiments, use of the methods, compositions, kits and systems of the present invention is directed by a biomarker of disease susceptibility and/or disease progression. In certain embodiments, the biomarker is a neuroimaging biomarker, a cerebrospinal fluid (CSF) biomarker, and/or a plasma biomarker. In other embodiments, the biomarker is a genomic biomarker, an epigenomic biomarker, a proteomic biomarker, a metabolomic biomarker, an exosomic biomarker, and/or a microbiomic biomarker.

### DESCRIPTION OF THE FIGURES

FIG. 1 shows α-syn monomer and PFF characterization. A) Human α-syn monomer and PFF were characterized by transmission electron microscopy (TEM). Scale bar, 100 nm. B) Distribution of human α-syn PFF length. Mean length of human PFF is 53.8 nm (n=237). C) Thioflavin T (ThT) assay of α-syn monomer and PFF. Quantification data are the means±SEM, n=3 independent experiments, Student's t test. ****P<0.0001.

FIG. 2 shows nanobody selection against α-syn PFF. A) Illustration of the disulfide bond-free nanobody library (based on PDB: 4LDE). C22 and C95(orange) that form the disulfide bond were replaced with C22L and C95A. The library was generated by randomizing residues on the 3 complementary determining regions, CDR 1, 2 and 3. Additionally, CDR3 was designed with 3 different loop length, 7, 11 and 15 amino acids. B) Schematic of PFF-nanobody selection. Nanobodies were expressed on the yeast surface by fusion to the C-terminus of the Aga2p mating protein, followed by a FLAG tag. Yeast cells that bind to α-syn PFF were selected using MACS and FACS. A representative selection gate (red trapezoid) is shown in the FACS plot. C) FACS analysis of PFF-Nanobody libraries before and after selection. After 6 rounds of selection, the selected nanobodies showed higher PFF-binding signal compared to nanobody library and negative control (without PFF). The numbers in the upper right Q2 indicate the yeast population ratio of Q2/Q4. D) FACS analysis of two nanobody clones PFFNB2 and PFFNB7.

FIG. 3 shows testing the binding of the disulfide bond-free nanobody on yeast surface. A) Schematics of the binding assay on the yeast-surface. Anti-GFP nanobody (GFPNB) with and without disulfide bond was expressed on the yeast surface following Aga2p. The yeast cells were incubated with EGFP-mCherry fusion protein. B) FACS analysis of yeast cells expressing GFPNB-FLAG on the surface incubated with EGFP-mCherry. GFPNB without a disulfide bond retains its binding affinity to EGFP. Negative control (−GFPNB), yeast cell without induction and no GFPNB expression.

FIG. 4 shows the nanobody library sequence and detailed selection results. A) Designed nanobody library sequences. The constant regions were determined from the consensus sequences of the VHH from llama gene IGHV1S1-IGHV1S1S5 (grey).³⁰ The three CDR loops (blue, green and red) and residues adjacent to the CDRs were determined to be highly variable by comparing 93 sequences of existing nanobodies, and therefore randomized in the libraries. The X sites (FIG. 4A) are randomized with 20 amino acids. The third loop was modified into 3 different lengths composed of 7, 11 and 15 amino acids (red). C22L and C95A mutation are indicated by orange letter. B) FACS analysis of the rounds of nanobody selection against α-syn PFF. After one round of MACS followed by 3 more rounds of FACS, a false positive nanobody population was observed that showed high fluorescence signal even in the absence of PFF incubation (post 4^{th} round). Removal of false positive clones on 5^{th} round using MACS failed (blue arrow, post 5^{th} round result). This is presumably because some low-expressing yeast cells can escape the negative selections using MACS, but will appear in the subsequent round after growth and re-amplification in the media. We used FACS to draw a tight gate to select only the true positive population but away from the false positive population (red triangle, post 5^{th} round result).

FIG. 5 shows FACS analysis of the 28 nanobody clones selected against α-syn PFF (PFFNB). Yeast cells expressing 28 different clones were incubated with α-syn PFF, monomer or buffer alone, followed by labelling with anti-α-syn primary antibody and secondary antibody conjugated to AlexaFluor 568. Numbers in the upper right Q2 indicate the ratio of Q2/Q4 population. All 28 clones showed selective binding to α-syn PFF, but not to α-syn monomers.

FIG. 6 shows analysis of the 3^{rd} CDR loops of the 28 PFFNB clones. A)₃rd loop length analysis of the 28 PFFNB clones. 47% of clones consists of 7 amino acids on the 3^{rd} loop, 32% consists of 11 amino acids and 21% consists of 15 amino acids. B) The 3^{rd} loops of selected PFFNB have a net positive charge and are rich in hydrophobic residues.

FIG. 7 shows SDS-PAGE analysis of the PFF nanobody expression and purification in E. coli. A) Protein expression in E. coli BL21. SDS-PAGE analysis of purified PFFNBs fused with HisTag and Maltose Binding Protein (MBP) at its N-terminus. Expected protein size is ˜60 kDa. The majority of protein at ˜60 kDa (green arrow) was retained in cell pellet while the extracted protein is at ˜50 kDa (red arrow). HisTag-MBP-GFPNB (positive control) was observed at the expected molecular weight (blue arrow). B) SDS-PAGE of HisTag-MBP-PFFNB or GFPNB expressed in E. coli BL21(C14) with chaperone co-expressed. All purified PFFNBs were observed at ˜60 kDa i.e., the expected molecular weight.

FIG. 8 shows in vitro characterization of PFFNB2 and PFFNB7. A) Native PAGE gel Western blot of the human α-syn monomer and PFF with PFFNB2 and 7, or anti-α-syn antibody. PFFNB2 and 7 bind selectively to the high molecular weight α-syn but not to the low molecular weight α-syn. Anti-α-syn antibody binds to both the high and low molecular weight α-syn forms. M, α-syn monomer. P, α-syn PFF. B) Imaging analysis of the colocalization of the intracellularly expressed PFFNB2, and C) PFFNB7 with the p129S-α-syn immunofluorescence. PFFNB2-GFP expressing HEK cells were transduced with or without PFF, followed by immunostaining with anti-p129S α-syn antibody (p129S-Ab) after 2 days of PFF transduction. White arrows indicate co-localization between GFP and p129S-Ab. Scale bars, 20 μm.

FIG. 9 shows PFFNB2 and 7 fused to GFP inhibit α-syn pathology induced by PFF in vitro. A) Wild-type (WT) mouse primary cortical neurons were treated with AAV expressing GFP, GFP-PFFNB2 or GFP-PFFNB7 at day-5 and α-syn PFF at day-7. The α-syn pathology level was assessed with anti-phosphorylated serine129 (pS129) immunostaining at day-7 after PFF treatment. Scale bar, 50 μm. B) Quantification of the pS129 immunoreactivity normalized by Hoechst. Quantification data are the means±SEM, n=6 independent experiments, one-way ANOVA followed by Tukey's correction. ****P<0.0001, ns, non-significant.

FIG. 10 shows PFFNB2 and 7 fused to GFP reduce α-syn pathology induced by PFF in vivo. A) Timeline of PFF animal experiments with AAV-PFFNBs treatment. Intracerebroventricular injection of AAV into the neonatal mouse brain was administered to Hu-SNCA WT mice. The mice were stereotaxically injected with PFF at 2-months old then sacrificed 1-month after PFF injection. B, C) pS129 immunostaining in the cortex normalized by Hoechst. Brain sections were stained with anti-pS129 antibody. Scale bar, 50 μm. D, E) Quantification of pS129 immunostaining in the cortex. Data are the means±SEM, n=4 mice per group, one-way ANOVA followed by Tukey's correction. ***P<0.001, ****P<0.0001, ns, non-significant.

FIG. 11 shows that PFFNBs fused to mCherry inhibit α-syn pathology induced by PFF in vitro. A) The WT mouse primary cortical neurons were treated with AAV expressing PFFNB2 and 7 fused to mCherry at 5 days and α-syn PFF at 7 days. The α-syn pathology level was assessed with anti-phosphorylated serine129 (pS129) immunostaining at 7 days after treatment. Scale bar, 50 μm. B) Quantification of inhibition of the pS129 immunoreactivity normalized by Hoechst and mCherry. Quantification data are the means±SEM, n=4 independent experiments, One-way ANOVA followed by Tukey's correction. *P<0.05, **P<0.01, ****P<0.0001

FIG. 12 shows α-syn pathology induced by PFF in the striatum in vivo. A) pS129 immunostaining in the striatum (ST). Brain sections were stained with anti-pS129 antibody. Scale bar, 50 μm. B) Quantification of pS129 immunostaining in the striatum. Data are means SEM, n=4 mice per group, one-way ANOVA followed by Tukey's correction. ns, non-significant.

### DEFINITIONS

To facilitate an understanding of the present disclosure, a number of terms and phrases are defined below:

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “non-human animals” refers to all non-human animals including, but not limited to, vertebrates such as rodents, non-human primates, ovines, bovines, ruminants, lagomorphs, porcines, caprines, equines, canines, felines, aves, etc.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., Alzheimer's disease, Parkinson's disease, atherosclerosis, cancer). Test compounds comprise both known and potential therapeutic compounds. A test compound can be determined to be therapeutic by screening using the screening methods of the present disclosure.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, and industrial samples. Such examples are not however to be construed as limiting the sample types applicable to the present disclosure.

As used herein, the term “effective amount” refers to the amount of a compound (e.g., a compound described herein) sufficient to effect beneficial or desired results. An effective amount can be administered in one or more administrations, applications or dosages and is not limited to or intended to be limited to a particular formulation or administration route.

As used herein, the term “co-administration” refers to the administration of at least two agent(s) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

As used herein, the term “pharmaceutical composition” refers to the combination of an active agent with a carrier, inert or active, making the composition especially suitable for diagnostic or therapeutic use in vivo, or ex vivo.

As used herein, the term “target binding agent” (e.g., “target-binding protein” or protein mimetic such as an aptamer) refers to proteins that bind to a specific target. “Target-binding proteins” include, but are not limited to, immunoglobulins, including polyclonal, monoclonal, chimeric, single chain, single domain, scFv, minibody, nanobody, and humanized antibodies, Fab fragments, F(ab′)2 fragments, and Fab expression libraries.

As used herein, the term “single-chain variable fragment” (scFv) refers to an antibody fragment that comprises a fusion protein of the variable regions of the heavy (V_{H}) and light chains (V_{L}) of an immunoglobulin. In some embodiments, the V_{H} and V_{L} are connected with a short linker peptide.

As used herein, the term “minibody” refers to an antibody fragment that retains antigen binding activity. In some embodiments, minibodies comprise an scFv fused to an Fc region (e.g., an IgG Fc region).

Various procedures known in the art are used for the production of polyclonal antibodies. For the production of antibodies, various host animals can be immunized by injection with the peptide or protein containing the desired epitope including but not limited to rabbits, mice, rats, sheep, goats, llamas, alpacas, etc. In a specific embodiment, the peptide is conjugated to an immunogenic carrier (e.g., diphtheria toxoid, bovine serum albumin (BSA), or keyhole limpet hemocyanin (KLH)). Various adjuvants are used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, Gerbu adjuvant and potentially useful human adjuvants such as BCG (Bacille Calmette-Guerin) and Corynebacterium parvum.

For preparation of monoclonal antibodies, any technique that provides for the production of antibody molecules by continuous cell lines in culture may be used (See e.g., Harlow and Lane, Antibodies: A Laboratory Manual, Cold Spring Harbor Laboratory Press, Cold Spring Harbor, NY). These include, but are not limited to, the hybridoma technique originally developed by Kohler and Milstein (Kohler and Milstein, Nature, 256:495-497 [1975]), as well as the trioma technique, the human B-cell hybridoma technique (See e.g., Kozbor et al., Immunol. Today, 4:72 [1983]), and the EBV-hybridoma technique to produce human monoclonal antibodies (Cole et al., in Monoclonal Antibodies and Cancer Therapy, Alan R. Liss, Inc., pp. 77-96 [1985]). In other embodiments, suitable monoclonal antibodies, including recombinant chimeric monoclonal antibodies and chimeric monoclonal antibody fusion proteins are prepared as described herein.

According to the invention, techniques described for the production of single chain antibodies (U.S. Pat. No. 4,946,778; herein incorporated by reference) can be adapted to produce specific single chain antibodies as desired. An additional embodiment of the invention utilizes the techniques known in the art for the construction of Fab expression libraries (e.g., Huse et al., Science, 246:1275-1281 [1989]) to allow rapid and easy identification of monoclonal Fab fragments with the desired specificity.

In some embodiments, monoclonal antibodies are generated using the ABL-MYC method (See e.g., U.S. Pat. Nos. 5,705,150 and 5,244,656, each of which is herein incorporated by reference) (Neoclone, Madison, WI). ABL-MYC is a recombinant retrovirus that constitutively expresses v-abl and c-myc oncogenes. When used to infect antigen-activated splenocytes, this retroviral system rapidly induces antigen-specific plasmacytomas. ABL-MYC targets antigen-stimulated (Ag-stimulated) B-cells for transformation.

In some embodiments, biopanning as described in Pardon et al., Nat Protoc. 2014 March; 9(3):674-93 is used to generate single domain antibodies. In some embodiments, to generate murine scFv units, phage-based biopanning strategies, of which there are several published protocols available, are used.

Antibody fragments that contain the idiotype (antigen binding region) of the antibody molecule can be generated by known techniques. For example, such fragments include but are not limited to: the F(ab′)2 fragment that can be produced by pepsin digestion of an antibody molecule; the Fab′ fragments that can be generated by reducing the disulfide bridges of an F(ab′)2 fragment, and the Fab fragments that can be generated by treating an antibody molecule with papain and a reducing agent.

Genes encoding target-binding proteins can be isolated by methods known in the art. In the production of antibodies, screening for the desired antibody can be accomplished by techniques known in the art (e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), Western Blots, precipitation reactions, agglutination assays (e.g., gel agglutination assays, hemagglutination assays, etc.), complement fixation assays, immunofluorescence assays, protein A assays, phage display biopanning, and immunoelectrophoresis assays, etc.).

As used herein, the term “toxic” refers to any detrimental or harmful effects on a cell or tissue as compared to the same cell or tissue prior to the administration of the toxicant.

### DETAILED DESCRIPTION OF THE DISCLOSURE

Provided herein are compositions and methods to prevent and to treat α-synucleinopathies for research and therapeutic purposes. In particular, provided herein are compositions, methods, kits and uses for inhibition of pathological phosphorylation and spread of α-synuclein in the central nervous system as therapeutic targets of neurodegenerative disorders including dementia with Lewy bodies and Parkinson's disease dementia.

Fibrillar α-syn aggregates are prion-like seeds able to propagate throughout the brain, and become a major driver in the pathogenesis⁸⁻¹⁰. It is crucial to design reagents targeting α-syn fibrillar form but not monomeric form. The reagents provide the opportunity to specifically inhibit α-syn pathology development in the pathogenesis. In experiments conducted in the course of development of certain embodiments of the present invention, we designed and generated disulfide bond-free nanobodies that specifically recognize α-syn fibrils in vitro and in cell lines. The disulfide-bond-free nanobodies are advantageous because they maintain the same folding under both oxidizing and reducing environments. We use the AAV viral expression system to support long-term intracellular expression of the disulfide bond-free nanobodies. Additionally, we have established the efficacy of exemplary AAV-PFFNB2-GFP and AAV-PFFNB7-GFP in inhibiting pathological phosphorylated α-syn in primary cortical neurons. In turn, we have discovered that intraventricular injection of agents such as AAV-PFFNB2-GFP and AAV-PFFNB7-GFP prevents the spread of α-syn pathology to the cortex in the striatal-PFF model. PD patients diagnosed with the onset of motor symptoms exhibit loss of dopaminergic neurons and substantial α-syn pathology in the substantia nigra (SN). As the disease progresses, pathogenic α-syn spreads from the SN to cortex, and ˜80% of PD develops cognitive impairment (CI) with α-syn pathology in the cortex⁴⁶⁻⁴⁸. As pathogenic α-syn spreads from the vagus nerve to the cortex, PD-CI is particularly debilitating because patients then suffer from delusions and hallucinations, together leading to significant cost in quality of life and increased mortality over PD alone. Thus, there is an urgent need to provide therapeutics to prevent the pathology spreading.

In experiments conducted in the course of development of certain embodiments of the present invention, we have determined that certain PFFNBs exhibit higher efficacy than nanobodies to α-syn monomer. Even though administration of antibodies targeting either α-syn monomer or aggregates could both prevent α-syn aggregation in mouse models^{20,38,39}, it is preferential to target the PFF only to minimize the functional perturbation of the physiological state of α-syn because α-syn monomer plays an important role in vesicle trafficking and refilling at the synapses. Depletion of α-syn results in imbalance between the reserved and releasable vesicles and impaired neurotransmitter uptake by the vesicles⁴³. Moreover, α-syn may form a tetramer that beneficially inhibits aggregation^{44,45} a formation that may be disrupted by nanobodies to α-syn monomer. Thus, it is preferred to target the pathogenic α-syn fibril form but not the monomer form.

Because α-synucleinopathies are long-term progressive disorders⁴⁰, conventional antibody administration may be expensive and inconvenient. Gene therapy is an alternative to frequent drug administration by enabling long-term expression of therapeutic proteins in the cells^{22,41}. Further, nanobodies are preferred over conventional antibodies or scFVs for intracellular applications because they are small, stable and expressed inside cells⁴². To maintain the identical folding under both oxidizing and reducing environments, we assembled disulfide bond-free synthetic nanobody libraries to select disulfide bond-free nanobodies against α-syn PFF for intracellular applications. Because the initial nanobody libraries are disulfide bond free, only nanobodies that fold properly without disulfide bonds and that bind to the α-syn PFF were selected. The disulfide bond-free nanobodies maintain the shared folding structure when expressed in the cytoplasm as on the cell surface necessary for the targeting of the α-syn fibrils intracellularly.

In experiments conducted in the course of development of certain embodiments of the present invention, we show that expression of the disulfide bond-free and α-syn PFF-selective nanobodies (e.g., PFFNB2 and 7) reduce α-syn pathology both in neuron cultures and mouse models. PFFNB2 and 7 provide instruments to test pathology development in different brain regions and their effects on behaviors and cognition. For example, AAV-PFFNB2 (or 7)-GFP may be introduced to different brain regions in PD mouse models to prevent the α-syn pathology development in that specific brain region. This permits investigation of the effects of α-syn pathology development prevention in specific brain regions on behaviors and cognition. PFFNBs find use as therapeutics for treating the α-syn pathology in PD patients. As well, our disulfide bond-free nanobody selection methods provide a newly developed strategy for designing protein binders that are stably expressed in the cytoplasm for targeting other diseased states of expressed proteins.

### Inhibitors and Agents

In some embodiments, the present disclosure provides α-syn PFF inhibitors and/agents. In certain embodiments, the α-syn PFF inhibitor and/or agent is a small molecule. Mimetics of binding agents are also provided. In other embodiments, the α-syn PFF inhibitor is an antibody. The present invention is not limited to the use of any particular antibody configuration. In given embodiments, the targeting unit is an antigen binding protein. Preferred antigen binding proteins include, but are not limited to an immunoglobulin, a Fab, F(ab′)₂, Fab′ single chain antibody, Fv, single chain (scFv), mono-specific antibody, bi-specific antibody, tri-specific antibody, multivalent antibody, chimeric antibody, humanized antibody, human antibody, CDR-grafted antibody, shark antibody, an immunoglobulin single variable domain (e.g., a nanobody also termed a single variable domain antibody), minibody, camelid antibody (e.g., from the Camelidae family) microbody, intrabody (e.g., intracellular antibody), and/or de-fucosylated antibody and/or derivative thereof. Nanobodies provide an alternative to scFV as an intrabody and offer multiple advantages (see above).

Further, the present invention also provides expression vectors comprising nucleic acid sequences encoding any of the above polypeptides or fusion proteins thereof or functional fragments thereof, as well as host cells expressing such expression vectors. Suitable expression systems include constitutive and inducible expression systems in bacteria or yeasts, virus expression systems, such as baculovirus, semliki forest virus and lentiviruses, or transient transfection in insect or mammalian cells. Suitable host cells include E. coli, Lactococcus lactis, Saccharomyces cerevisiae, Schizosaccharomyces pombe, Pichia pastoris, and the like. Suitable animal host cells include HEK 293, COS, S2, CHO, NSO, DT40 and the like. The cloning, expression and/or purification of the antibodies can be done according to techniques known by the skilled person in the art.

It will be understood that polypeptides described herein may be identified with reference to the nucleotide and/or amino acid sequence corresponding to the variable and/or complementarity determining regions (“CDRs”) thereof.

Also within the scope of the invention are natural or synthetic analogs, mutants, variants, alleles, homologs and orthologs (herein collectively referred to as “variants”) of the immunoglobulin single variable domains of the invention as defined herein. Thus, according to one embodiment of the invention, the term “immunoglobulin single variable domain of the invention” in its broadest sense also covers such variants, in particular variants of the antibodies described herein. Generally, in such variants, one or more amino acid residues may have been replaced, deleted and/or added compared to the antibodies of the invention as defined herein. Such substitutions, insertions or deletions may be made in one or more of the framework regions and/or in one or more of the CDRs. Variants, as used herein, are sequences wherein each or any framework region and each or any complementarity determining region shows at least 80% identity, preferably at least 85% identity, more preferably 90% identity, even more preferably 95% identity or, still even more preferably 99% identity with the corresponding region in the reference sequence (e.g., FR1_variant versus FR1_reference, CDR1_variant versus CDR1_reference, FR2 variant versus FR2_reference, CDR2_variant versus CDR2_reference, FR3_variant versus FR3_reference, CDR3_variant versus CDR3_reference, FR4_variant versus FR4_reference), as can be measured electronically by making use of algorithms such as PILEUP and BLAST. (See, e.g., Higgins & Sharp, CABIOS 5:151 (1989); Altschul S. F., W. Gish, W. Miller, E. W. Myers, D. J. Lipman. Basic local alignment search tool. J. Mol. Biol. 1990; 215:403-10.) Software for performing BLAST analyses is publicly available through the National Center for Biotechnology Information (on the worldwide web at ncbi.nlm.nih.gov/). Such variants of immunoglobulin single variable domains may be of particular advantage since they may have improved potency or other desired properties.

A “deletion” is defined here as a change in either amino acid or nucleotide sequence in which one or more amino acid or nucleotide residues, respectively, are absent as compared to an amino acid sequence or nucleotide sequence of a parental polypeptide or nucleic acid. Within the context of a protein, a deletion can involve deletion of about two, about five, about ten, up to about twenty, up to about thirty or up to about fifty or more amino acids. A protein or a fragment thereof may contain more than one deletion.

An “insertion” or “addition” is that change in an amino acid or nucleotide sequences which has resulted in the addition of one or more amino acid or nucleotide residues, respectively, as compared to an amino acid sequence or nucleotide sequence of a parental protein. “Insertion” generally refers to addition to one or more amino acid residues within an amino acid sequence of a polypeptide, while “addition” can be an insertion or refer to amino acid residues added at an N- or C-terminus, or both termini. Within the context of a protein or a fragment thereof, an insertion or addition is usually of about one, about three, about five, about ten, up to about twenty, up to about thirty or up to about fifty or more amino acids. A protein or fragment thereof may contain more than one insertion.

A “substitution,” as used herein, results from the replacement of one or more amino acids or nucleotides by different amino acids or nucleotides, respectively as compared to an amino acid sequence or nucleotide sequence of a parental protein or a fragment thereof. It is understood that a protein or a fragment thereof may have conservative amino acid substitutions which have substantially no effect on the protein's activity. By conservative substitutions is intended combinations such as gly, ala; val, ile, leu, met; asp, glu; asn, gln; ser, thr; lys, arg; cys, met; and phe, tyr, trp.

By means of non-limiting examples, a substitution may, for example, be a conservative substitution (as described herein) and/or an amino acid residue may be replaced by another amino acid residue that naturally occurs at the same position in another variable domain. Thus, any one or more substitutions, deletions or insertions, or any combination thereof, that either improve the properties of the antibody of the invention or that at least do not detract too much from the desired properties or from the balance or combination of desired properties of the antibody of the invention (i.e., to the extent that the antibody is no longer suited for its intended use) are included within the scope of the invention. A skilled person will generally be able to determine and select suitable substitutions, deletions or insertions, or suitable combinations of thereof, based on the disclosure herein and optionally after a limited degree of routine experimentation, which may, for example, involve introducing a limited number of possible substitutions and determining their influence on the properties of the antibodies thus obtained.

Further, depending on the host organism used to express the immunoglobulin single variable domain of the invention, such deletions and/or substitutions may be designed in such a way that one or more sites for post-translational modification (such as one or more glycosylation sites) are removed, as will be within the ability of the person skilled in the art. Alternatively, substitutions or insertions may be designed so as to introduce one or more sites for attachment of functional groups (as described herein), for example, to allow site-specific pegylation.

Examples of modifications, as well as examples of amino acid residues within the immunoglobulin single variable domain, that can be modified (e.g., either on the protein backbone but preferably on a side chain), methods and techniques that can be used to introduce such modifications and the potential uses and advantages of such modifications will be clear to the skilled person. For example, such a modification may involve the introduction (e.g., by covalent linking or in another suitable manner) of one or more functional groups, residues or moieties into or onto the immunoglobulin single variable domain of the invention, and in particular of one or more functional groups, residues or moieties that confer one or more desired properties or functionalities to the immunoglobulin single variable domain of the invention. Examples of such functional groups and of techniques for introducing them will be clear to the skilled person, and can generally comprise all functional groups and techniques mentioned in the general background art cited hereinabove as well as the functional groups and techniques known per se for the modification of pharmaceutical proteins, and in particular for the modification of antibodies or antibody fragments (including ScFvs and single domain antibodies), for which reference is, for example, made to Remington's Pharmaceutical Sciences, 16th ed., Mack Publishing Co., Easton, Pa. (1980). Such functional groups may, for example, be linked directly (for example, covalently) to an immunoglobulin single variable domain of the invention, or optionally via a suitable linker or spacer, as will again be clear to the skilled person. One of the most widely used techniques for increasing the half-life and/or reducing immunogenicity of pharmaceutical proteins comprises attachment of a suitable pharmacologically acceptable polymer, such as poly(ethyleneglycol) (PEG) or derivatives thereof (such as methoxypoly(ethyleneglycol) or mPEG). Generally, any suitable form of pegylation can be used, such as the pegylation used in the art for antibodies and antibody fragments (including but not limited to (single) domain antibodies and ScFvs); reference is made to, for example, Chapman, Nat. Biotechnol., 54, 531-545 (2002); by Veronese and Harris, Adv. Drug Deliv. Rev. 54, 453-456 (2003), by Harris and Chess, Nat. Rev. Drug. Discov., 2, (2003) and in WO04060965. Various reagents for pegylation of proteins are also commercially available, for example, from Nektar Therapeutics, USA. Preferably, site-directed pegylation is used, in particular via a cysteine-residue (see, for example, Yang et al., Protein Engineering, 16, 10, 761-770 (2003). For example, for this purpose, PEG may be attached to a cysteine residue that naturally occurs in an antibody of the invention, an antibody of the invention may be modified so as to suitably introduce one or more cysteine residues for attachment of PEG, or an amino acid sequence comprising one or more cysteine residues for attachment of PEG may be fused to the N- and/or C-terminus of an antibody of the invention, all using techniques of protein engineering known per se to the skilled person. Preferably, for the immunoglobulin single variable domains and proteins of the invention, a PEG is used with a molecular weight of more than 5000, such as more than 10,000 and less than 200,000, such as less than 100,000; for example, in the range of 20,000-80,000. Another, usually less preferred modification comprises N-linked or O-linked glycosylation, usually as part of co-translational and/or post-translational modification, depending on the host cell used for expressing the immunoglobulin single variable domain or polypeptide of the invention. Another technique for increasing the half-life of an immunoglobulin single variable domain may comprise the engineering into bifunctional constructs or into fusions of immunoglobulin single variable domains with peptides (for example, a peptide against a serum protein such as albumin).

Yet another modification may comprise the introduction of one or more detectable labels or other signal-generating groups or moieties, depending on the intended use of the labeled antibody. Suitable labels and techniques for attaching, using and detecting them will be clear to the skilled person and, for example, include, but are not limited to, fluorescent labels (such as fluorescein, isothiocyanate, rhodamine, phycoerythrin, phycocyanin, allophycocyanin, o-phthaldehyde, and fluorescamine and fluorescent metals such as Eu or others metals from the lanthanide series), phosphorescent labels, chemiluminescent labels or bioluminescent labels (such as luminal, isoluminol, theromatic acridinium ester, imidazole, acridinium salts, oxalate ester, dioxetane or GFP and its analogs), radio-isotopes, metals, metals chelates or metallic cations or other metals or metallic cations that are particularly suited for use in in vivo, in vitro or in situ diagnosis and imaging, as well as chromophores and enzymes (such as malate dehydrogenase, staphylococcal nuclease, delta-V-steroid isomerase, yeast alcohol dehydrogenase, alpha-glycerophosphate dehydrogenase, triose phosphate isomerase, biotinavidin peroxidase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholine esterase). Other suitable labels will be clear to the skilled person and, for example, include moieties that can be detected using NMR or ESR spectroscopy. Such labeled antibodies and polypeptides of the invention may, for example, be used for in vitro, in vivo or in situ assays (including immunoassays known per se such as ELISA, RIA, EIA and other “sandwich assays,” etc.), as well as in vivo diagnostic and imaging purposes, depending on the choice of the specific label. As will be clear to the skilled person, another modification may involve the introduction of a chelating group, for example, to chelate one of the metals or metallic cations referred to above. Suitable chelating groups, for example, include, without limitation, diethyl-enetriaminepentaacetic acid (DTPA) or ethylenediaminetetraacetic acid (EDTA). Yet another modification may comprise the introduction of a functional group that is one part of a specific binding pair, such as the biotin-(strept)avidin binding pair. Such a functional group may be used to link the antibody of the invention to another protein, polypeptide or chemical compound that is bound to the other half of the binding pair, i.e., through formation of the binding pair. For example, an antibody of the invention may be conjugated to biotin, and linked to another protein, polypeptide, compound or carrier conjugated to avidin or streptavidin. For example, such a conjugated antibody may be used as a reporter, for example, in a diagnostic system where a detectable signal-producing agent is conjugated to avidin or streptavidin. Such binding pairs may, for example, also be used to bind the antibody of the invention to a carrier, including carriers suitable for pharmaceutical purposes. One non-limiting example are the liposomal formulations described by Cao and Suresh, Journal of Drug Targeting, 8, 4, 257 (2000). Such binding pairs may also be used to link a therapeutically active agent to the antibody of the invention.

In some embodiments, the immunoglobulin single variable domain of the present invention is fused to a detectable label, either directly or through a linker. Preferably, the detectable label is a radio-isotope or radioactive tracer, which is suitable for medical applications, such as in in vivo nuclear imaging. Examples include, without the purpose of being limitative, ⁹⁹mTc, ¹²³I, ¹²⁵I, ¹¹¹In, ¹⁸F, ⁶⁴Cu, ⁶⁷Ga, ⁶⁸Ga, and any other radio-isotope which can be used in animals, in particular mouse or human.

In still another embodiment, the immunoglobulin single variable domain of the present invention is fused to a moiety selected from the group consisting of a toxin, or to a cytotoxic drug, or to an enzyme capable of converting a prodrug into a cytotoxic drug, or to a radionuclide, or coupled to a cytotoxic cell, either directly or through a linker.

In some embodiments, the present invention provides an antibody-drug conjugate and/or an antibody-enzyme conjugate. In certain embodiments, the antibody drug conjugates are administered to cells expressing α-syn PFF.

As used herein, “linkers” are peptides of 1 to 50 amino acids length and are typically chosen or designed to be unstructured and flexible. These include, but are not limited to, synthetic peptides rich in Gly, Ser, Thr, Gln, Glu or further amino acids that are frequently associated with unstructured regions in natural proteins. (See, e.g., Dosztanyi Z., V. Csizmok, P. Tompa, and I. Simon (2005). IUPred: web server for the prediction of intrinsically unstructured regions of proteins based on estimated energy content. Bioinformatics (Oxford, England), 21(16), 3433-4.)

In certain embodiments, the immunoglobulin is a recombinant immunoglobulin, a subunit, or an antigen binding fragment thereof (e.g., has a variable region, or at least a complementarity determining region (CDR)).

In some embodiments, the immunoglobulin is monovalent (e.g., includes one pair of heavy and light chains, or antigen binding portions thereof). In other embodiments, the immunoglobulin is a divalent (e.g., includes two pairs of heavy and light chains, or antigen binding portions thereof).

In some embodiments, inhibitors and agents of the present invention comprise 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity with one or more of SEQ ID Nos: 1-28. In other embodiments, inhibitors and agents of the present invention comprise at least 15, 16, 17, 18, 19, 20, 21, 22, 23, 24 or 25 contiguous amino acids of one or more of SEQ ID Nos: 1-28. In further embodiments, inhibitors and agents of the present invention are 70%, 71%, 72%, 73%, 74%, 75%, 76%, 77%, 78%, 79%, 80%, 81%, 82%, 83%, 84%, 85%, 86%, 87%, 88%, 89%, or 90% identical to one or more of SEQ ID Nos: 1-28. In some embodiments, the present invention provides a therapy that comprises an inhibitor or agent comprising 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity with one or more of SEQ ID Nos: 1-28 in combination with a therapy for α-synucleinopathy comprising, for example, a drug (e.g., levodopa, carbidopa, safinamide, pramipexole, rotigotine, and ropinirole, amantadine, benztropine, trihexyphenidyl, selegiline, rasagileine, entacaptone and/or tolcaptone), a small molecule, a second nanobody, a nucleic acid, an aptamer, and/or a drug that treats side effects and/or idiosyncratic reactions to one or more components if the combination therapy.

### Gene Transfer

The present disclosure contemplates the use of any genetic manipulation for use in modulating the expression of α-syn PFF. Examples of genetic manipulation include, but are not limited to, heterologous expression of the agents described herein, gene knockout (e.g., removing the α-syn PFF pathway genes from the chromosome using, for example, recombination), CRISPR, expression of antisense constructs with or without inducible promoters, and the like. Delivery of nucleic acid construct to cells in vitro or in vivo may be conducted using any suitable method. A suitable method is one that introduces the nucleic acid construct into the cell such that the desired event occurs (e.g., expression of an antisense construct or nanobody).

Introduction of molecules carrying genetic information into cells is achieved by any of various methods including, but not limited to, directed injection of naked DNA constructs, bombardment with gold particles loaded with said constructs, and macromolecule mediated gene transfer using, for example, liposomes, biopolymers, and the like. Exemplary methods use gene delivery vehicles derived from viruses, including, but not limited to, adenoviruses, retroviruses, vaccinia viruses, and adeno-associated viruses (AAV). Because of the higher efficiency as compared to retroviruses, vectors derived from adenoviruses are the preferred gene delivery vehicles for transferring nucleic acid molecules into host cells in vivo. Adenoviral vectors have been shown to provide very efficient in vivo gene transfer into a variety of tissues in animal models. Examples of adenoviral and AAV vectors and methods for gene transfer are described in PCT publications WO 00/12738 and WO 00/09675 and U.S. Pat. Nos. 6,033,908, 6,019,978, 6,001,557, 5,994,132, 5,994,128, 5,994,106, 5,981,225, 5,885,808, 5,872,154, 5,830,730, and 5,824,544, each of which is herein incorporated by reference in its entirety.

Vectors may be administered to subject in a variety of ways. For example, in some embodiments of the present disclosure, vectors are administered into central nervous system (CNS) tissues, and/or into the cerebrospinal fluid (CSF). In other embodiments, administration is via the blood or lymphatic circulation (See e.g., PCT publication 1999/02685 herein incorporated by reference in its entirety). Exemplary dose levels of adenoviral vector are preferably 10⁸ to 10¹¹ vector particles added to the perfusate. See, for example, Bulcha, J. T., Wang, Y., Ma, H. et al. Viral vector platforms within the gene therapy landscape Sig Transduct Target Ther 6, 53 (2021).

### CNS Delivery

In some embodiments, the α-syn PFF inhibitors and agents are delivered to the CNS by methods and compositions that promote transfer across the blood brain barrier (BBB). In certain embodiments, the methods and compositions comprise one or more bi-specific immunoglobulins comprising, for example, immunoglobulins to highly expressed proteins, including basigin, Glut1, and CD98hc. Immunoglobulins to these targets are significantly enriched in the brain after administration in vivo. In particular, immunoglobulins against CD98hc show robust accumulation in brain after systemic dosing. Accordingly, in specific embodiments, methods and compositions of the present invention comprise, for example, use of CD98hc as a robust receptor-mediated transcytosis pathway for immunoglobulin delivery to the brain. (Zuchero et al. Neuron 89; 70-82, 2016.) In further embodiments, transfer across the BBB is enhanced by transient disruption, for example, osmotic or pharmacologic disruption, and/or by other membrane protein pathways using receptor-mediate transcytosis comprising, for example, antibodies against the transferrin receptor.

### Pharmaceutical Compositions and Formulations

The present disclosure further provides pharmaceutical compositions (e.g., comprising the compounds described above). The pharmaceutical compositions of the present disclosure may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral, intravenous or parenteral. Parenteral administration includes intravenous, intra-arterial, subcutaneous, intraperitoneal or intramuscular injection or infusion; or intracranial, e.g., intrathecal or intraventricular, administration. In certain embodiments, α-syn PFF inhibitors and agents are administered by methods that bypass the BBB including, for example, direct application to the surface of the CNS, to the parenchyma of the CNS, to the ventricles of the CNS, and to the cerebrospinal fluid (CSF) of the CNS. In particular, intrathecal and epidural administration may be achieved by single shot, a series of single shots, and/or by continuous administration to the CSF. In certain embodiments, continuous administration to the CSF is provided by a programmable external pump. In other embodiments, continuous administration is provided by a programmable implantable pump.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present disclosure include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present disclosure, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present disclosure may be formulated into any of many possible dosage forms such as, but not limited to, tablets, capsules, liquid syrups, soft gels, suppositories, and enemas. The compositions of the present disclosure may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

Agents that enhance uptake of oligonucleotides at the cellular level may also be added to the pharmaceutical and other compositions of the present disclosure. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present disclosure may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present disclosure, such as dyes, flavoring agents, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC50s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligonucleotide is administered in maintenance doses, ranging from 0.01 μg to 100 g per kg of body weight, once or more daily, to once every 20 years.

### Experimental Examples

The following examples are provided in order to demonstrate and further illustrate certain embodiments and aspects of the present disclosure and are not to be construed as limiting the scope thereof.

### Production of α-Syn Monomer and PFF

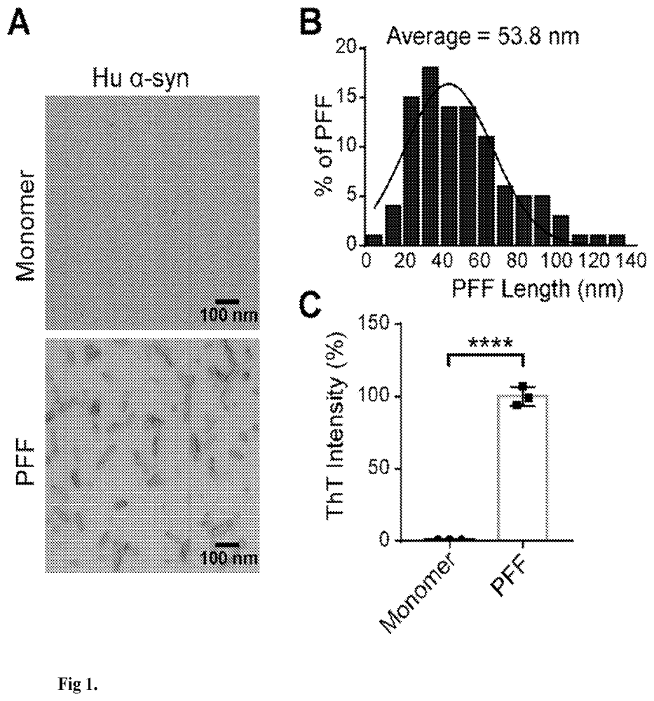

In experiments conducted in the course of development of certain embodiments of the present invention, to design nanobodies that preferentially bind to the α-syn PFF but not the monomer, we prepared both α-syn monomer and PFF following an established protocol^{13,30}. Seven days after agitation we generated mature fibrils from α-syn monomer, and we further sonicated the fibrils into α-syn PFF. Transmission electron microscopy (TEM) showed the short fibril (average length 53.8 nm) of α-syn PFF (FIG. 1), and no regular structure of α-syn monomer (FIG. 1A). α-syn PFF was further confirmed with thioflavin T (ThT) fluorescence assay (FIG. 1C).

### Designing Disulfide Bond-Free Synthetic Nanobodies for α-Syn PFF

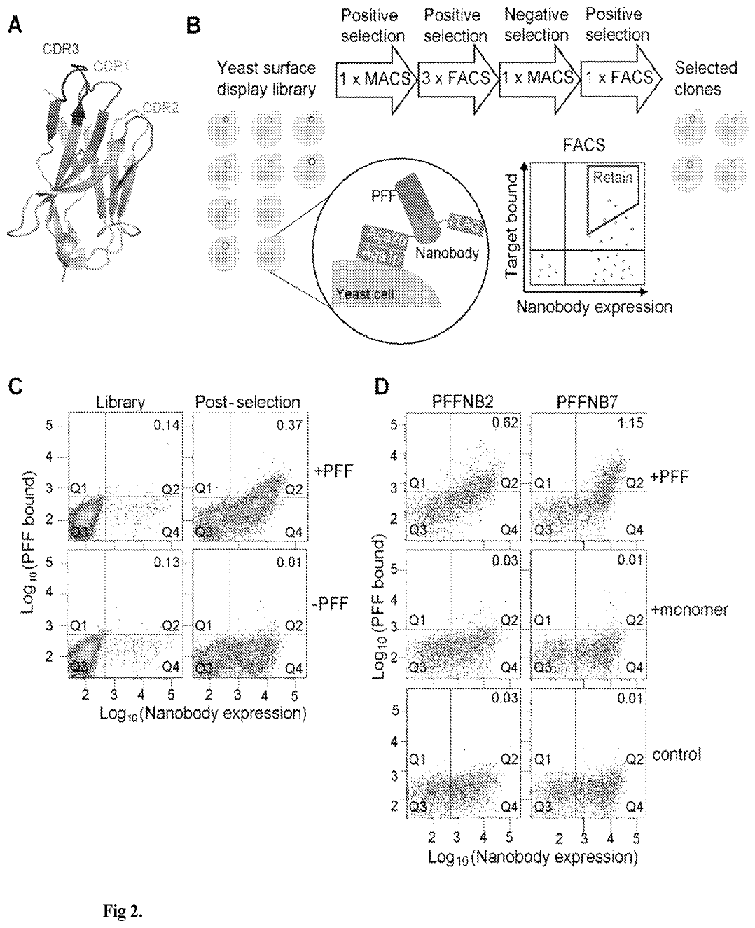

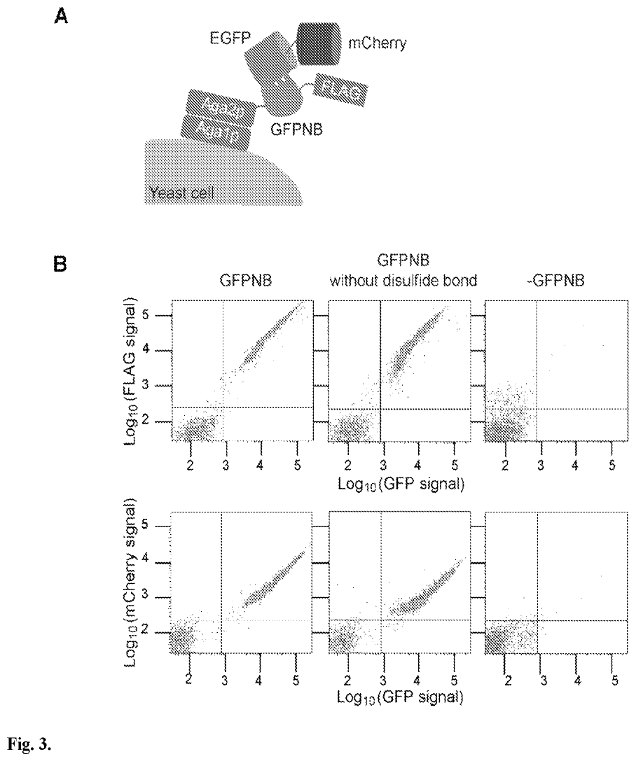

Nanobodies have a common structure that has β-barrel constant regions (colored in grey, FIG. 2A) and 3 variable loops that form the complementary determining regions (CDRs, colored in blue, green and red, FIG. 2A). The highly variable CDRs constitute the antigen binding site.

Under oxidizing conditions in, for example, the secretory pathway and the extracellular environment, 2 cysteine residues inside the β-barrel form a disulfide bond (orange line, FIG. 2A)^{31,32}, thereby stabilizing the nanobody scaffold. However, in the reducing environment of the cytosol, the disulfide bond is reduced to cysteine residues, causing structural differences for the nanobody under the oxidizing and reducing environment. Because the synthetic nanobodies are designed against α-syn PFF for intracellular applications, we remove the disulfide bond to ensure that the selected nanobodies have the same structural folding in the reducing cytosolic environment as in the oxidizing extracellular environment.

In experiments conducted in the course of development of certain embodiments of the present invention, to evaluate whether the nanobody folds correctly without the disulfide bond, we introduced C22L and C95A mutations into GFP nanobody (GFPNB)³³. FIG. 3 shows that the disulfide bond-free GFPNB binds to EGFP on yeast cell surface. This indicates that the nanobody folds correctly with these two cysteine mutations. Therefore, we constructed the disulfide-bond free nanobody libraries with C22L and C95A mutations introduced. The CDRs were constructed following the protocol in a previous synthetic nanobody selection study (FIG. 2A, 4A).³⁰ The nanobody libraries were displayed on the yeast surface via fusion to Aga2p, a mating protein on the yeast surface (FIG. 2B).

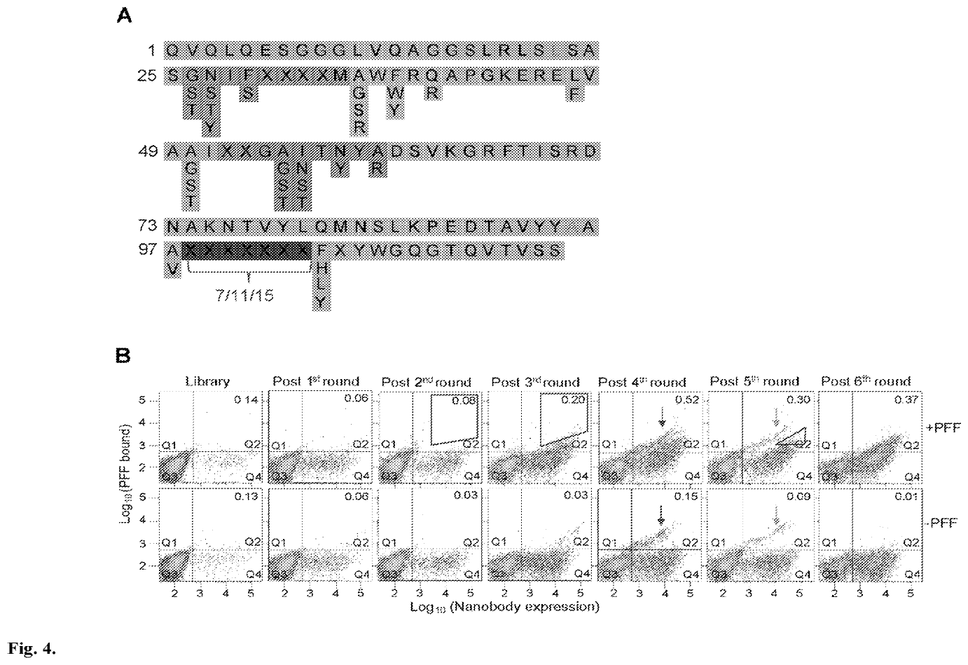

Next, we performed disulfide bond-free nanobody selection against α-syn PFF. As illustrated in FIG. 2B, yeast cell libraries with nanobodies on the surface were first treated with α-syn PFF. Then, the α-syn PFF bound to the nanobodies were characterized with anti-α-syn antibodies. The yeast cells were further labelled with secondary antibody conjugated with either magnetic beads for magnetic-activated cell sorting (MACS), or fluorophores for fluorescence-activated cell sorting (FACS). Nanobodies that bind to PFF were selected in the first round of sorting using MACS to allow screening of a large population of yeast cells (up to 10⁹ cells). From the MACS enriched clones, we performed 5 more rounds of FACS and MACS (FIG. 2B) to enrich nanobodies that can bind to α-syn PFF (FIG. 2C). Details of the selection process are provided in FIG. 4.

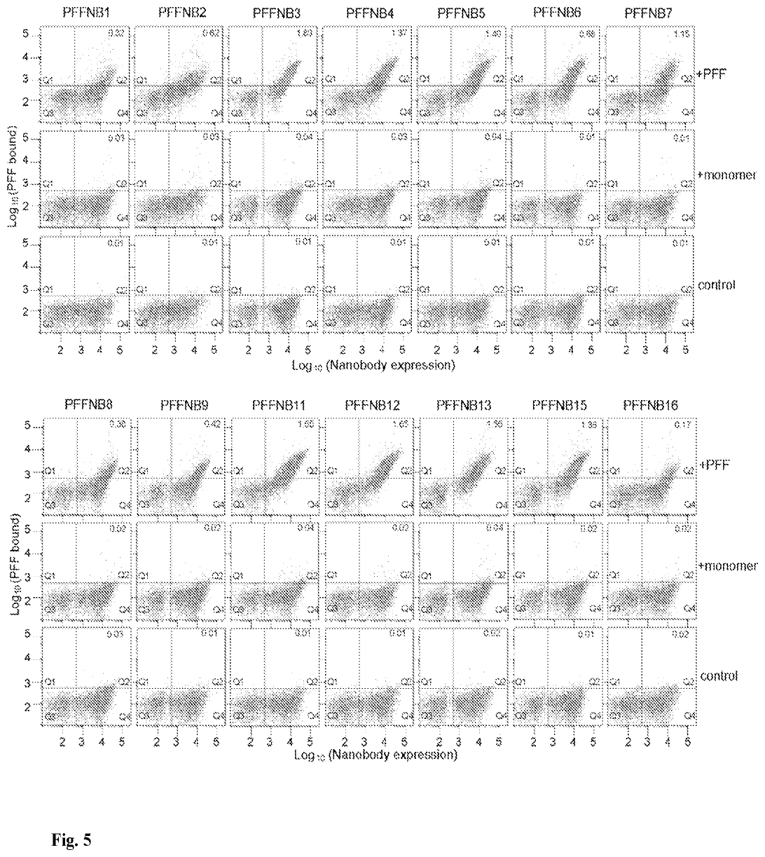

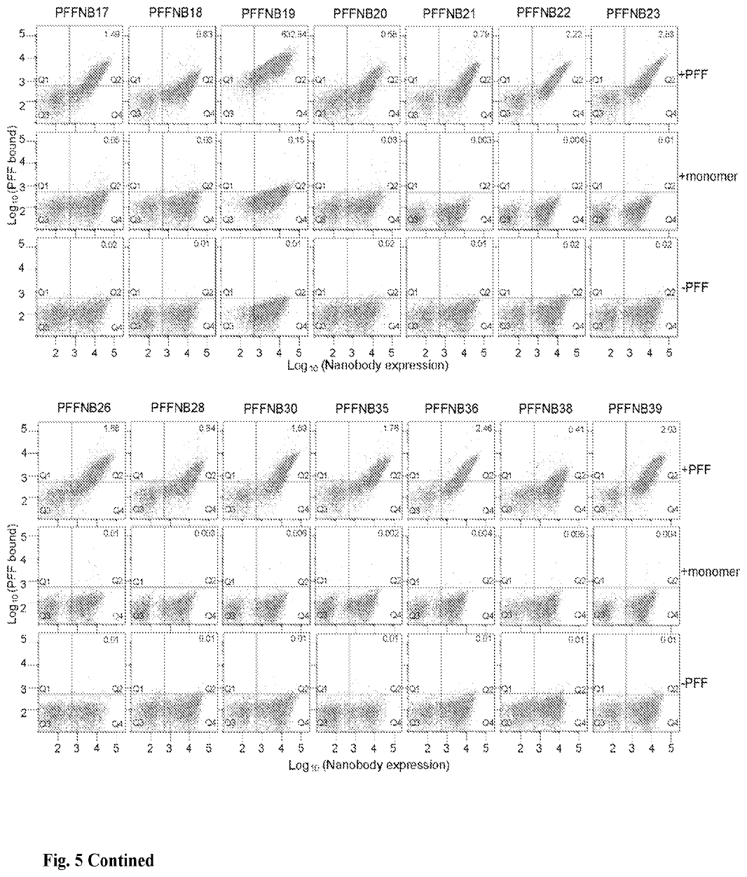

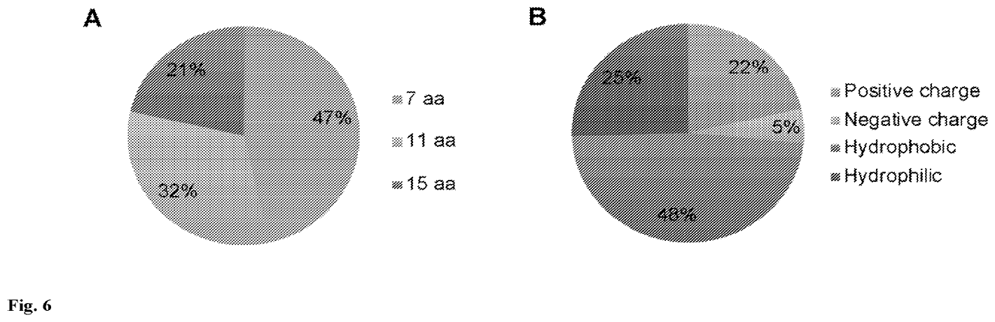

From the enriched yeast cells, we extracted the plasmid DNA and retransformed the DNA into bacteria cells for individual clone sequencing. From the 40 nanobody clones sequenced, 28 unique clones were identified. Twenty-eight clones were bound preferentially to the α-syn PFF over the monomer (Table Si, FIG. 5) referred to as PFF-nanobodies (PFFNBs). Of the clones, 47% comprised 7 amino acids in the third loop (FIG. 6). The selected nanobodies are rich in hydrophobic and positively-charged residues in the third loop (FIG. 6B), that complement the α-syn aggregate surfaces because α-syn is rich in hydrophobic residues where they aggregate,³⁵ and also rich in negatively charged residues at its c-terminus³⁶.

### In Vitro Characterization and Validation of the Selected PFFNBs

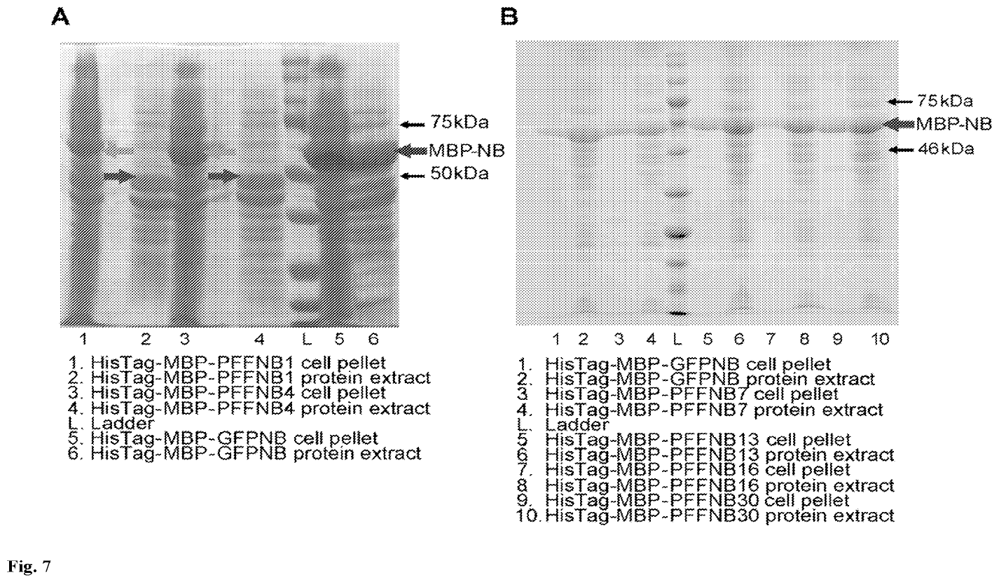

In experiments conducted in the course of development of certain embodiments of the present invention, to further validate our PFFNBs' binding to α-syn PFF, we constructed PFFNBs with maltose binding protein (MBP) fusion at its N-terminus for protein expression in E. coli (BL21). The nanobody proteins were retained in the cell pellet when expressed in E. coli (BL21) but not the positive control GFPNB(C22L, C95A), indicating that the PFFNBs are less stable than GFPNB(C22L, C95A) (FIG. 7A). Successful expression of soluble PFFNBs was achieved in the cytosol of the BL21(C14) E. coli cells that are supplemented with a chaperon protein (plasmid pGro7) (FIG. 7B).

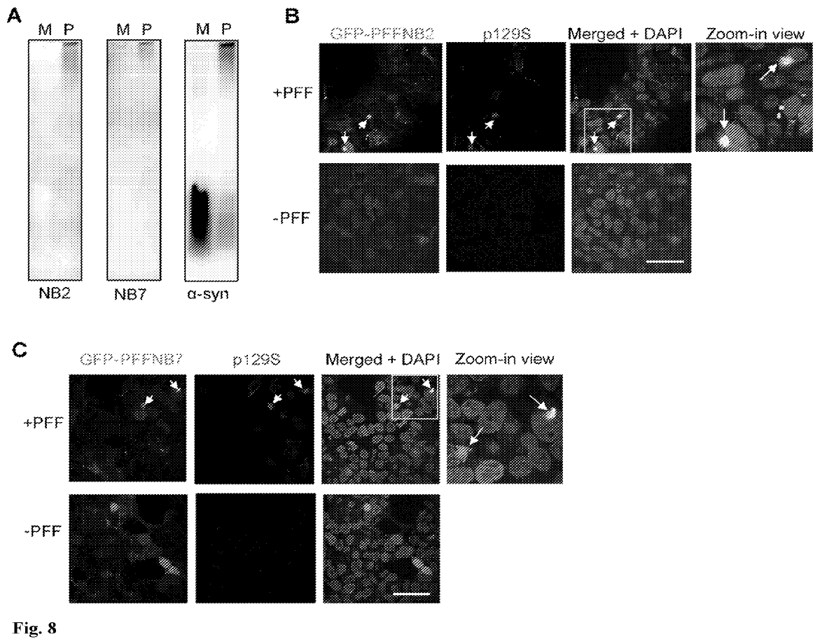

Purified MBP-PFFNBs were used to immunoblot against α-syn monomer and PFF following native polyacrylamide gel electrophoresis (PAGE). Anti-α-syn antibody was used as a control. FIG. 8A shows that PFFNB2 and PFFNB7 selectively recognized the α-syn PFF but not the monomer, while commercial anti-α-syn antibody could detect both the low molecular weight α-syn monomer and the high molecular weight α-syn aggregate (FIG. 8A).

In experiments conducted in the course of development of certain embodiments of the present invention, we evaluated the expression and binding of PFFNB2 and PFFNB7 to α-syn PFF in the cytosol of mammalian cells. To produce pathological α-syn PFF in HEK293T cells, we followed an established protocol³⁵. BEK cells expressing both A53T-α-syn monomer and PFFNBs were transduced α-syn PFF. Two days later, the α-syn PFF propagates in the A53T-α-syn expressing HEK cells. The cells were fixed and immune-stained to evaluate colocalization between the PFFNBs and the phosphorylated α-syn. FIGS. 8B and 8C show that the level of phosphorylated serine 129 (pS129) α-syn increased 2 days after α-syn PFF transduction, consistent with previous studies.⁴⁶ Phosphorylated serine129 (pS129) α-syn is a typical pathological marker in PD patients^{12,37}. Additionally, the GFP-PFFNB2 and GFP-PFFNB7 fusion proteins co-localized with the phosphorylated α-syn puncta induced by α-syn PFF transduction. These data show that PFFNB2 and 7 both bind recombinant α-syn PFF and pathological phosphorylated serine129 (pS129) α-syn formed in mammalian cells, and further indicate the correct folding and functions of the PFFNBs when expressed intracellularly.

### PFFNbs Inhibited α-Syn Pathology Induced by PFF in Primary Cortical Neurons

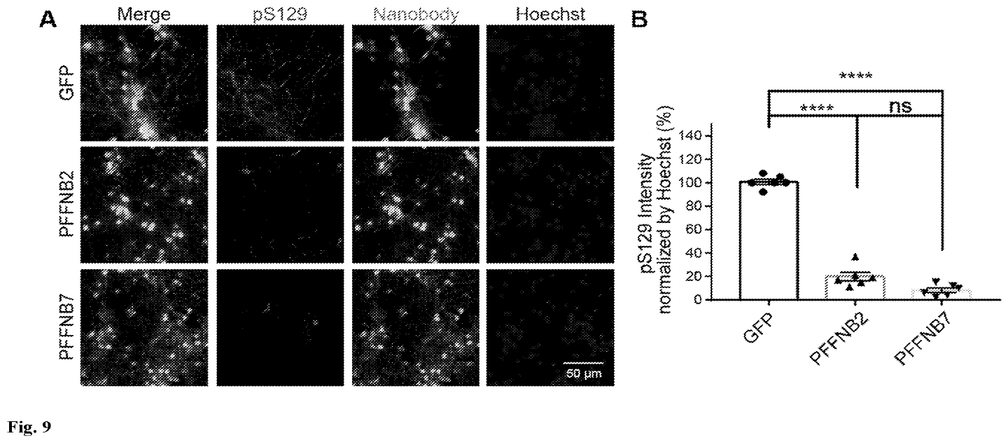

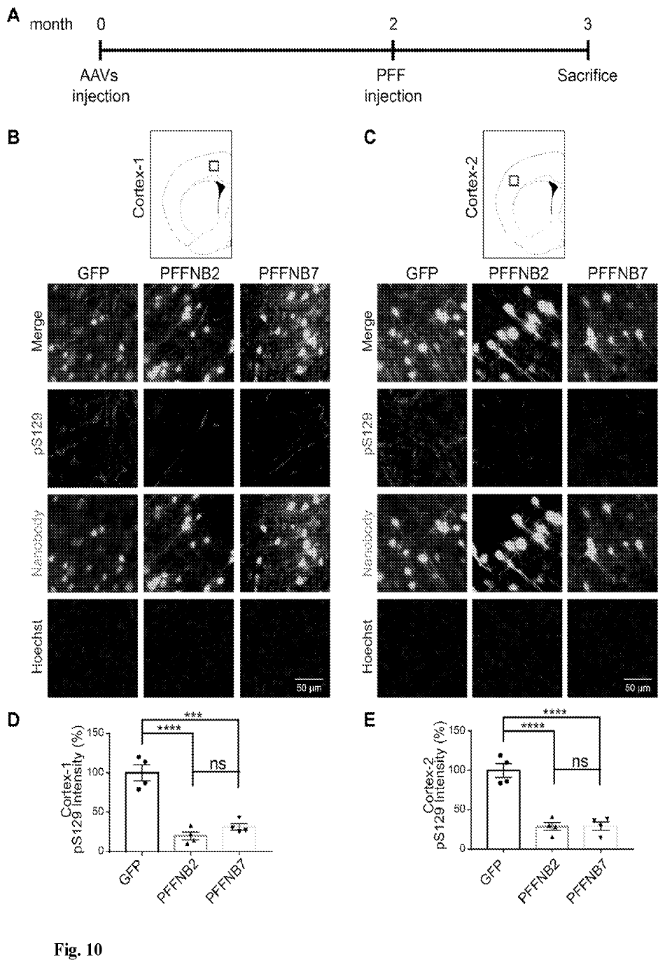

In experiments conducted in the course of development of certain embodiments of the present invention, to determine whether PFFNBs inhibit α-syn pathology in vitro, GFP, GFP-PFFNBs and mCherry-PFFNBs fusion DNA were cloned into AAV vector with synapsin promoter. To produce AAV, 3XT150 flasks of HEK293T cells (<20 passages, 100% confluency) was each transfected with 5.2 μg AAV vector, 4.35 μg AAV1, 4.35 μg AAV2 serotype plasmids and 10.4 μg pDF6 adenovirus helper plasmid with PEI (Polyethylenimine) transfection. After 36 to 48 hours incubation at 37° C. under 5% C02, cells were collected and AAV were concentrated following previous publication.⁴⁹ AAV-PFFNB2-GFP and AAV-PFFNB7-GFP were individually added to the primary cortical neuron cultures at 5 days in vitro (DIV), followed by the administration of PFF at 7 DIV. AAV-GFP was used as the control group. Immunoreactivity of anti-pS129 was observed in cortical neurons 7 days after PFF administration as published¹¹. We observed that immunoreactivity of anti-pS129 was significantly decreased in both AAV-PFFNB2 and AAV-PFFNB7 treatment groups, compared to AAV-GFP (FIG. 9A, 9B). We also compared the efficiency of the PFFNBs to NbSyn87 that targets the α-syn monomer in preventing α-syn PFF-induced pathology in primary cortical neurons (FIG. 10). PFFNB2 and 7 both showed superior efficacy than NbSyn87 in decreasing the immunoreactivity of anti-pS129, indicating that α-syn PFF is an effective target in inhibiting development of induced by pathogenic α-syn.

### PFFNBs Prevented α-Syn Pathology Spreading to the Cortex Induced by Intrastriatal Injection of PFF In Vivo

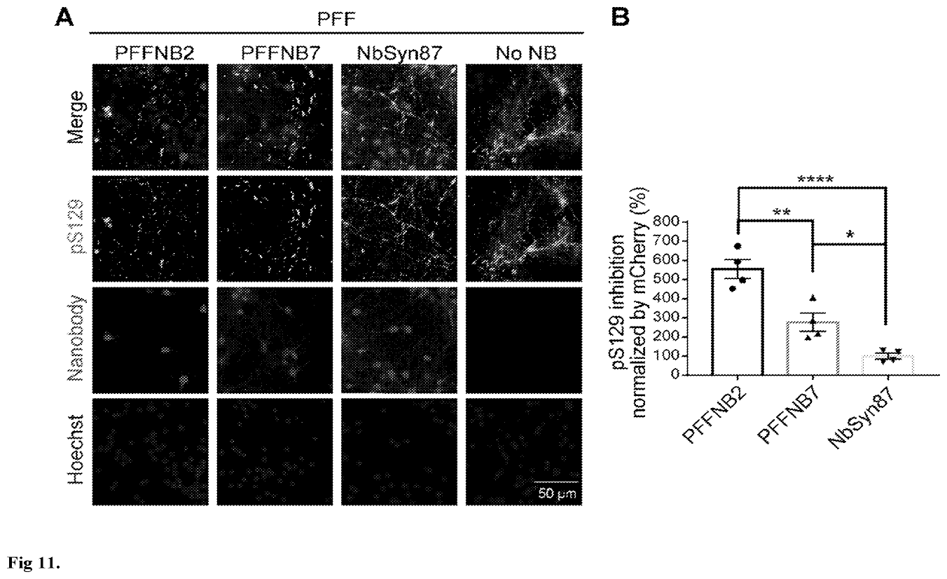

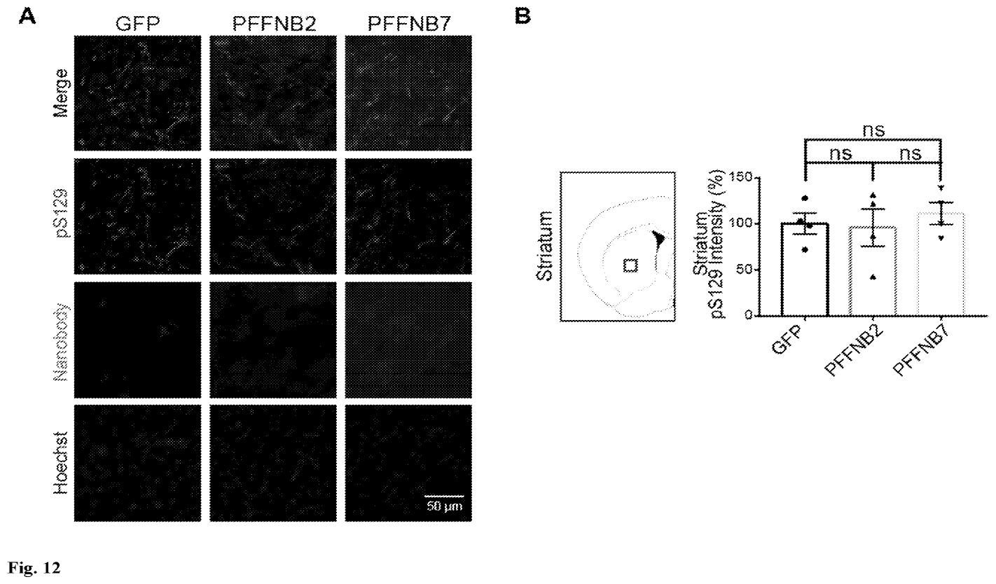

In experiments conducted in the course of development of certain embodiments of the present invention, to further determine whether PFFNB2 and PFFNB7 can prevent α-syn pathology spreading in vivo, we performed the intracerebroventricular injection of AAV-PFFNB2-GFP, AAV-PFFNB7-GFP and AAV-GFP to neonatal transgenic mice harboring a Snca knockout and a transgene encoding the human α-syn (Jackson Laboratory, strain No. 010710). Two months after AAV injection, we then performed the intrastriatal injection of α-syn PFF to the mice (FIG. 11A). Intrastriatal injection of α-syn PFF induces α-syn pathology spreading to the cortex 1 month after injection. To determine whether AAV-PFFNBs-GFP inhibits α-syn pathology spreading to the cortex, we assessed the immunoreactivity of anti-pS129 of the cortex of the mice 1 month after injection (FIG. 11A). Intracerebroventricular injection of AAV-PFFNBs-GFP with synapsin promoter resulted in PFFNBs-GFP expression in neurons of the cerebral cortex, consistent with the published study.⁵⁰ Two cortical sub-regions were chosen with dense labelling and high intensity expression of GFP. Substantial immunoreactivity of anti-pS129 was observed (FIG. 11B, 11C), indicating α-syn pathology spreading from the striatum to the cortex in GFP control group. In contrast, significant reduction of pS129 level were detected in groups with intraventricular injection of AAV-PFFNB2-GFP and AAV-PFFNb7-GFP, indicating AAV-PFFNBs prevent α-syn pathology spreading to the cortex (FIG. 11D, 11E). There was no significant reduction of pS129 level in the striatum by AAV-PFFNBs-GFP, most probably attributable to little appreciable GFP expression signal in the striatum (FIG. 12A, 12B). These data indicate that intracerebroventricular injection of AAV-PFFNBs-GFP effectively blocks propagation of pathogenic α-syn to the cortex in vivo.

TABLE S1
Amino acid sequences of identified 28 PFFNB clones

| Nanobody | Sequences |
|---|---|
| PFFNB1 | QVQLQESGGGLVQAGGSLRLSLSASRYIFLLQKMGWYRQAPGKERELVAG |
| | IHKGSDTNYGDSVKGRFTLSRDNAKNTVYLQMNSLKSDDTAVYYAAEPVP |
| | PRPRRRPPLPYWGQGTQVTVSS (SEQ ID NO: 3) |
| PFFNB2 | QVQLQESGGGLVQAGGSLRLSLSASRYIFTLMGMRWYRRAPGKERELVASI |
| | QVGSDTNYRDSVKGRFTLSRDNAKNTVYLQMNSLKSDDTAVYYAAAPAY |
| | ARRLHRYWGQGTQVTVSS (SEQ ID NO: 1) |
| PFFNB3 | QVQLQESGGGLVQAGGSLRLSLSASRYISFLKLMGWFRRAPGKERELVAGI |
| | HNGTNTNYRDSVKGRFTLSRDNAKNTVYLQMNSLKSDDTAVYYAAEPQP |
| | LWTITVTRREHPYDYWGQGTQVTVSS (SEQ ID NO: 4) |
| PFFNB4 | QVQLQESGGGLVQAGGSLRLSPSASWYISRWWGMGWFRRAPGKERELVA |
| | SIDPGGDTNYPDSVKGRFTLSRDNAKNTVYLQMNSLKSDDTAVYYAAAAH |
| | PKLPTSPGKFQYWGQGTQVTVSS (SEQ ID NO: 5) |
| PFFNB5 | QVQLQESGGGLVQAGGSLRLSLSASRYIFLLPLMGWFRRAPGKERELVAGI |
| | ARGTTTYYPDSVKGRFTLSRDNAKNTVYLQMNSLKSDDTAVYYAAAHRV |
| | TKPQATKPFRYWGQGTQVTVSS (SEQ ID NO: 6) |
| PFFNB6 | QVQLQESGGGLVQAGGSLRLSLSASRNIFWWPLMGWYRRAPGKEREFVAS |
| | INSGTNTYYRDSVKGRFTLSRDNAKNTVYLQMNSLKSDDTAVYYAAAPFP |
| | APETHTYWGQGTQVTVSS (SEQ ID NO: 7) |
| PFFNB7 | QVQLQESGGGLVQAGGSLRLSLSASRYIFFWPGMGWFRRAPGKERELVASI |
| | PTGGATYYRDSVKGRFTLSRDNAKNTVYLQMNSLKSDDTAVYYAAAPPRP |
| | PATDKPLLPYWGQGTQVTVSS (SEQ ID NO: 2) |
| PFFNB8 | QVRLQESGGGLVQAGGSLRLSLSASGNIFRWWKMGWYRRAPGKERELVA |
| | SIQTGANTYYADSVKGRFTLSRDNAKNTVYLQMNSLKSDDTAVYYAAEW |
| | LPSSQKNPYPHPYWGQGTQVTVSS (SEQ ID NO: 8) |
| PFFNB9 | QVQLQESGGGLVQAGGSLRLSLSASGSIFQWWAMRWFRRAPGKERELVAS |
| | IHRGTVTNYPDSVKGRFTLSRDNAKNTVYLQMNSLKSDDTAVYYAAASTP |
| | DLSQNLLTDGLHRYWGQGTQVTVSS (SEQ ID NO: 9) |
| PFFNB11 | QVQLQESGGGLVQAGGSLRLSLSASRNIFLWLPMGWFRRAPGKEREFVASI |
| | GTGAATNYGDSVKGRFTLSRDNAKNTVYLQMNSLKSDDTAVYYAAARLA |
| | PHHRYSYWGQGTQVTVSS (SEQ ID NO: 10) |
| PFFNB12 | QVQLQESGGGLVQAGGSLRLSLSASRYIFPYLFMGWFRRAPGKERELVASI |
| | AAGTDTNYRDSVKGRFTLSRDNAKNTVYLQMNSLKSDDTAVYYAAARRP |
| | PKQTYLYWGQGTQVTVSS (SEQ ID NO: 11) |
| PFFNB13 | QVQLQESGGGLVQAGGCLRLSLSASRYIFHWCPMRWYRRAPGKERELVAS |
| | IPRGGATYYRDSVKGRFTLSRDNAKNTVYLQMNSLKSDDTAVYYAAAHQ |
| | AHPSPTATRYKYWGQGTQVTVSS (SEQ ID NO: 12) |
| PFFNB15 | QVQLQESGGGLVQAGGSLRLSLSASRYIFPWIVMGWFRRAPGKEREFVASI |
| | NSGSTTNYRDSVKGRFTLSRDNAKNTVYLQMNSLKSDDTAVYYAAAQKE |
| | GRTTARTQINTYYGYWGQGTQVTVSS (SEQ ID NO: 13) |
| PFFNB16 | QVQLQESGGGLVQAGGSLRLSLSASRYIFAPRWMRWFRRAPGKERELVAG |
| | IQFGGDTNYGDSVKGRFTLSRDNAKNTVYLQMNSLKSDDTAVYYAAETLY |
| | YPRANPRPLPYWGQGTQVTVSS (SEQ ID NO: 14) |
| PFFNB17 | QVQLQESGGGLVQAGGSLRLSLSASRYIFLYPLMGWFRRAPGKEREFVASI |
| | RRGTVTNYPDSVKGRFTLSRDNAKNTVYLQMNSLKSDDTAVYYAAATGN |
| | DRCCNRLGKQMFHPYWGQGTQVTVSS (SEQ ID NO: 15) |
| PFFNB18 | QVQLQESGGGLVQAGGSLRLSLSASRYIFVWAPMGWYRRAPGKERELVAS |
| | IKAGTNTYYRDSVKGRFTLSRDNAKNTVYLQMNSLKSDDTAVYYAAAAPP |
| | DETRTVLTITNDHNYWGQGTQVTVSS (SEQ ID NO: 16) |
| PFFNB19 | QVQLQESGGGLVQAGGSLRLSLSASRYIFFWPGMGWFRRAPGKERELVASI |
| | PTGGATYYRDSVKGRFTLSRDNAKNTVYLQMNSLKSDDTAVYYAAAPPRP |
| | PATDKPLLPYWGQGTQVTVSS (SEQ ID NO: 17) |
| PFFNB20 | QVQLQESGGGLVQAGGSLRLSLSASRYISWLQGMRWFRRAPGKEREFVASI |
| | IPGTNTNYGDSVKGRFTLSRDNAKNTVYLQMNSLKSDDTAVYYAAERRGQ |
| | PLLHPYWGQGTQVTVSS (SEQ ID NO: 18) |
| PFFNB21 | QVQLQESGGGLVQAGGSLRLSLSASRYIFLPLFMGWYRRAPGKERELVAGI |
| | NRGTTTNYPDSVKGRFTLSRDNAKNTVYLQMNSLKSDDTAVYYAAAAPS |
| | QTQYHKYWGQGTQVTVSS (SEQ ID NO: 19) |
| PFFNB22 | QVQLQESGGGLVQAGGSLRLSLSASRNIFSWMIMRWYRRAPGKERELVAG |
| | IKSGTDTNYRDSVKGRFTLSRDNAKNTVYLQMNSLKSDDTAVYYAAATPT |
| | QPTPHGYWGQGTQVTVSS (SEQ ID NO: 20) |
| PFFNB23 | QVQLQESGGGLVQAGGSLRLSLSASRTIFYWLGMRWFRRAPGKERELVAG |
| | IRTGATTNYRDSVKGRFTLSRDNAKNTVYLQMNSLKSDDTAVYYAAAPSV |
| | PPAYHPYWGQGTQVTVSS (SEQ ID NO: 21) |
| PFFNB26 | QVQLQESGGGLVQAGGSLRLSLSASRYIFVLPWMRWYRRAPGKERELVASI |
| | TDGATTNYPDSVKGRFTLSRDNAKNTVYLQMNSLKSDDTAVYYAAEVNR |
| | PRQAYGYWGQGTQVTVSS (SEQ ID NO: 22) |
| PFFNB28 | QVQLQESGGGLVQAGGSLRLSLSASRSIFRCRYMRWFRQAPGKERELVASII |
| | CGTNTNYRDSVKGRFTLSRDNAKNTVYLQMNSLKSDDTAVYYAAETTEA |
| | HLRPVTNLMYWGQGTQVTVSS (SEQ ID NO: 23) |
| PFFNB30 | QVQLQESGGGLVQAGGSLRLSLSASRYIFWGPFMGWFRRAPGKERELVAGI |
| | AHGSNTNYADSVKGRFTLSRDNAKNTVYLQMNSLKSDDTAVYYAAAQAP |
| | VHPHHPYWGQGTQVTVSS (SEQ ID NO: 24) |
| PFFNB35 | QVQLQESGGGLVQAGGSLRLSLSASRYIFAWYRMGWFRRAPGKERELVAS |
| | IQHGTITNYADSVKGRFTLSRDNAKNTVYLQMNSLKSDDTAVYYAAAQLG |
| | APRQYRYWGQGTQVTVSS (SEQ ID NO: 25) |
| PFFNB36 | QVQLQESGGGLVQAGGSLRSLSASWNIFHFEWMRWFRRAPGKEREFVASI |
| | HRGSNTNYPDSVKGRFTLSRDNAKNTVYLQMNSLKSDDTAVYYAAAKSPF |
| | TVPLTYWGQGTQVTVSS (SEQ ID NO: 26) |
| PFFNB38 | QVQLQESGGGLVQAGGSLRLSLSASRSISWRGWMGWFRRAPGKEREFVAS |
| | IGPGTNTNYADSVKGRFTLSRDNAKNTVYLQMNSLKSDDTAVYYAAEHLI |
| | PYYRFSYWGQGTQVTVSS (SEQ ID NO: 27) |
| PFFNB39 | QVQLQESGGGLVQAGGSLRLSLSASRSIFWWLGGMGWYRQAPGKERELV |
| | AGIAKGGNTNYRDSVKGRFTLSRDNAKNTVYLQMNSLKSDDTAVYYAAA |
| | QRGKSMRPLPTRRLSHSYWGQGTQVTVSS (SEQ ID NO: 28) |

### REFERENCES

1. Baba, M. et al. Aggregation of alpha-synuclein in Lewy bodies of sporadic Parkinson's disease and dementia with Lewy bodies. Am. J. Pathol. 152, 879-84 (1998).
2. Dodel, R. et al. Lewy body dementia and Parkinson's disease with dementia. J. Neurol. 255, 39-47 (2008).
3. Arnaoutoglou, N. A., O'Brien, J. T. & Underwood, B. R. Dementia with Lewy bodies from scientific knowledge to clinical insights. Nat. Rev. Neurol. 15, 103-112 (2019).
4. Olichney, J. M. et al. Cognitive decline is faster in Lewy body variant than in Alzheimer's disease. Neurology 51, 351-357 (1998).
5. Wong, Y. C. & Krainc, D. α-synuclein toxicity in neurodegeneration: mechanism and therapeutic strategies. Nat. Med 23, 1-13 (2017).
6. Braak, H. & Braak, E. Pathoanatomy of Parkinson's disease. J. Neurol. 247, 113-10 (2000).
7. Braak, H. et al. Staging of brain pathology related to sporadic Parkinson's disease. Neurobiol. Aging 24, 197-211 (2003).
8. Li, J.-Y. et al. Lewy bodies in grafted neurons in subjects with Parkinson's disease suggest host-to-graft disease propagation. Nat. Med 14, 501-503 (2008).
9. Li, W. et al. Extensive graft-derived dopaminergic innervation is maintained 24 years after transplantation in the degenerating parkinsonian brain. Proc. Natl. Acad Sci. 113, 6544-6549 (2016).
10. Kordower, J. H., Chu, Y., Hauser, R. A., Freeman, T. B. & Olanow, C. W. Lewy body-like pathology in long-term embryonic nigral transplants in Parkinson's disease. Nat. Med 14, 504-506 (2008).
11. Volpicelli-Daley, L. A. et al. Exogenous α-Synuclein Fibrils Induce Lewy Body Pathology Leading to Synaptic Dysfunction and Neuron Death. Neuron 72, 57-71 (2011).
12. Luk, K. C. et al. Pathological-Synuclein Transmission Initiates Parkinson-like Neurodegeneration in Nontransgenic Mice. Science (80-.). 338, 949-953 (2012).
13. Mao, X. et al. Pathological-synuclein transmission initiated by binding lymphocyte-activation gene 3. Science (80-.). 353, aah3374-aah3374 (2016).
14. Kam, T.-I. et al. Poly(ADP-ribose) drives pathologic α-synuclein neurodegeneration in Parkinson's disease. Science (80-.). 362, eaat8407 (2018).
15. Liu, Y.-Q. et al. Nanozyme scavenging ROS for prevention of pathologic α-synuclein transmission in Parkinson's disease. Nano Today 36, 101027 (2021).
16. Kovacs, G. G. et al. An antibody with high reactivity for disease-associated α-synuclein reveals extensive brain pathology. Acta Neuropathol. 124, 37-50 (2012).
17. Fagerqvist, T. et al. Monoclonal antibodies selective for α-synuclein oligomers/protofibrils recognize brain pathology in Lewy body disorders and α-synuclein transgenic mice with the disease-causing A30P mutation. J. Neurochem. 126, 131-144 (2013).
18. Vaikath, N. N. et al. Generation and characterization of novel conformation-specific monoclonal antibodies for α-synuclein pathology. Neurobiol. Dis. 79, 81-99 (2015).
19. Covell, D. J. et al. Novel conformation-selective alpha-synuclein antibodies raised against different in vitro fibril forms show distinct patterns of Lewy pathology in Parkinson's disease. Neuropathol. Appl. Neurobiol. 43, 604-620 (2017).
20. Weihofen, A. et al. Development of an aggregate-selective, human-derived α-synuclein antibody BIIB054 that ameliorates disease phenotypes in Parkinson's disease models. Neurobiol. Dis. 124, 276-288 (2019).
21. Emadi, S., Barkhordarian, H., Wang, M. S., Schulz, P. & Sierks, M. R. Isolation of a Human Single Chain Antibody Fragment Against Oligomeric α-Synuclein that Inhibits Aggregation and Prevents α-Synuclein-induced Toxicity. J. Mol. Biol. 368, 1132-1144 (2007).
22. Messer, A. & Joshi, S. N. Intrabodies as Neuroprotective Therapeutics. Neurotherapeutics 10, 447-458 (2013).
23. Hamers-Casterman, C. et al. Naturally occurring antibodies devoid of light chains. Nature 363, 446-448 (1993).
24. Jank, L. et al. Current Approaches and Future Perspectives for Nanobodies in Stroke Diagnostic and Therapy. Antibodies 8, 5 (2019).
25. Dong, J.-X. et al. A toolbox of nanobodies developed and validated for use as intrabodies and nanoscale immunolabels in mammalian brain neurons. Elife 8, (2019).
26. Vuchelen, A. et al. 1H, 13C and 15N assignments of a camelid nanobody directed against human α-synuclein. Biomol. NAMR Assign. 3, 231-233 (2009).
27. Guilliams, T. et al. Nanobodies Raised against Monomeric α-Synuclein Distinguish between Fibrils at Different Maturation Stages. J. Mol. Biol. 425, 2397-2411 (2013).
28. Butler, D. C. et al. Bifunctional anti-non-amyloid component α-Synuclein nanobodies are protective in situ. PLoS One 11, (2016).
29. Chatterjee, D. et al. Proteasome-targeted nanobodies alleviate pathology and functional decline in an α-synuclein-based Parkinson's disease model. npj Park. Dis. 4, 1-10 (2018).
30. McMahon, C. et al. Yeast surface display platform for rapid discovery of conformationally selective nanobodies. Nat. Struct. Mol. Biol. 25, 289-296 (2018).
31. Gelpi, E. et al. Multiple organ involvement by alpha-synuclein pathology in Lewy body disorders. Mov. Disord. 29, 1010-1018 (2014).
32. Ito, S. et al. Alpha-synuclein immunohistochemistry of gastrointestinal and biliary surgical specimens for diagnosis of Lewy body disease. Int. J. Clin. Exp. Pathol. 7, 1714-23 (2014).
33. Goldman, J. G. et al. Cognitive impairment in Parkinson's disease: a report from a multidisciplinary symposium on unmet needs and future directions to maintain cognitive health. npj Park. Dis. 4, 19 (2018).
34. Masliah, E. et al. Effects of α-Synuclein Immunization in a Mouse Model of Parkinson's Disease. Neuron 46, 857-868 (2005).
35. Jankovic, J. et al. Safety and Tolerability of Multiple Ascending Doses of PRX002/RG7935, an Anti-α-Synuclein Monoclonal Antibody, in Patients With Parkinson Disease. JAMA Neurol. 75, 1206 (2018).
36. Lashuel, H. A., Overk, C. R., Oueslati, A. & Masliah, E. The many faces of α-synuclein: from structure and toxicity to therapeutic target. Nat. Rev. Neurosci. 14, 38-48 (2013).
37. Bartels, T., Choi, J. G. & Selkoe, D. J. α-Synuclein occurs physiologically as a helically folded tetramer that resists aggregation. Nature 477, 107-110 (2011).
38. Wang, W. et al. A soluble-synuclein construct forms a dynamic tetramer. Proc. Natl. Acad. Sci. 108, 17797-17802 (2011).
39. Goedert, M., Spillantini, M. G., Del Tredici, K. & Braak, H. 100 years of Lewy pathology. Nat. Rev. Neurol. 9, 13-24 (2013).
40. Bulaklak, K. & Gersbach, C. A. The once and future gene therapy. Nat. Commun. 11, 5820 (2020).
41. Muyldermans, S. Nanobodies: Natural Single-Domain Antibodies. Annu. Rev. Biochem. 82, 775-797 (2013).
42. Volpicelli-Daley, L. A., Luk, K. C. & Lee, V. M. Y. Addition of exogenous α-synuclein preformed fibrils to primary neuronal cultures to seed recruitment of endogenous α-synuclein to Lewy body and Lewy neurite-like aggregates. Nat. Protoc. 9, 2135-2146 (2014).
43. Hagihara, Y., Mine, S. & Uegaki, K. Stabilization of an Immunoglobulin Fold Domain by an Engineered Disulfide Bond at the Buried Hydrophobic Region. J. Biol. Chem. 282, 36489-36495 (2007).
44. Kunz, P. et al. The structural basis of nanobody unfolding reversibility and thermoresistance. Sci. Rep. 8, 7934 (2018).
45. Kirchhofer, A. et al. Modulation of protein properties in living cells using nanobodies. Nat. Struct. Mol. Biol. 17, 133-139 (2010).
46. Luk, K. C. et al. Exogenous α-synuclein fibrils seed the formation of Lewy body-like intracellular inclusions in cultured cells. Proc. Natl. Acad. Sci. U.S.A 106, 20051-20056 (2009).
47. Binolfi, A. et al. Interaction of α-Synuclein with Divalent Metal Ions Reveals Key Differences: A Link between Structure, Binding Specificity and Fibrillation Enhancement. J. Am. Chem. Soc. 128, 9893-9901 (2006).
48. Fujiwara, H. et al. α-Synuclein is phosphorylated in synucleinopathy lesions. Nat. Cell Biol. 4, 160-164 (2002).
49. Wang, W. et al. A light- and calcium-gated transcription factor for imaging and manipulating activated neurons. Nat. Biotechnol. 35, 864-871 (2017).
50. Kim, J.-Y., Grunke, S. D., Levites, Y., Golde, T. E. & Jankowsky, J. L. Intracerebroventricular Viral Injection of the Neonatal Mouse Brain for Persistent and Widespread Neuronal Transduction. J. Vis. Exp. (2014). doi:10.3791/51863

All publications and patents mentioned in the above specification are herein incorporated by reference. Various modifications and variations of the described method and system of the disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure that are obvious to those skilled relevant fields are intended to be within the scope of the following claims.

We claim:

1. A method of treating an α-synucleinopathy, comprising exposing cells of a subject's central nervous system (CNS) to an α-synuclein preformed fibrils (PFF) inhibitor wherein said exposing treats said α-synucleinopathy.

2. The method of claim 1, wherein said subject is a human subject.

3. The method of claim 1, wherein said α-synucleinopathy is Lewy body dementia (LBD), Parkinson's disease with dementia (PDD) and/or dementia with Lewy bodies (DLB).

4. The method of claim 1, wherein said α-synuclein preformed fibrils (PFF) inhibitor is an anti-α-synuclein preformed fibrils (PFF) antibody.

5. The method of claim 4, wherein said anti-α-synuclein preformed fibrils (PFF) antibody is an anti-α-synuclein preformed fibrils (PFF) nanobody.

6. The method of claim 5, wherein said anti-α-synuclein preformed fibrils (PFF) nanobody does not comprise a disulfide bond.

7. The method of claim 5, wherein said anti-α-synuclein preformed fibrils (PFF) nanobody is fibril-specific.

8. The method of claim 5, wherein said anti-α-synuclein preformed fibrils (PFF) nanobody is encoded into an adeno-associated virus (AAV) vector.

9. The method of claim 8, wherein said anti-α-synuclein preformed fibrils (PFF) nanobody is encoded by PFFNB2 (SEQ ID NO. 1) and/or PFFNB7 (SEQ ID NO. 2).

10. The method of claim 8, wherein said AAV vector is administered by intracerebroventricular administration.

11. The method of claim 1, wherein said α-synuclein preformed fibrils (PFF) inhibitor interferes with α-synuclein preformed fibrils (PFF) expression.

12. The method of claim 1, wherein said exposing is in vivo exposing, ex vivo exposing or in vitro exposing.

13. The method of claim 1, wherein said exposing to said α-synuclein preformed fibrils (PFF) inhibitor is selected from the group consisting of local administration, topical administration, intrathecal administration, intraparenchymal administration, intracerebroventrical administration, intravenous administration, intraarterial administration, intrapulmonary administration and oral administration.

14. The method of claim 1, wherein said exposing comprises combination therapy with an agent that treats α-synucleinopathy.

15. The method of claim 14, wherein said agent is selected from the group consisting of a small molecule, a peptide, and a nucleic acid.

16. A method of treating an α-synucleinopathy disease in a subject, comprising:
a. assaying a plasma and/or cerebrospinal fluid (CSF) sample from a subject, and
b. administering an agent that is an α-synuclein preformed fibrils (PFF) inhibitor.

17. A composition comprising:
a) α-synuclein preformed fibrils (PFF) inhibitor; and
b) a pharmaceutically acceptable carrier.

18. The composition of claim 17, wherein said anti-α-synuclein preformed fibrils (PFF) antibody is an anti-α-synuclein preformed fibrils (PFF) nanobody.

19. The composition of claim 18, wherein said anti-α-synuclein preformed fibrils (PFF) nanobody is encoded into an adeno-associated virus (AAV) vector.

20. The composition of claim 17, wherein said anti-α-synuclein preformed fibrils (PFF) nanobody is encoded by PFFNB2 (SEQ ID NO. 1) and/or PFFNB7 (SEQ ID NO. 2).

21. Use of a composition of any of claims 17-20.

22. Use of a composition of any of claims 17-20 for the treatment of a disease in a subject.


SEQUENCE LISTING (28 sequences)

SEQ ID NO: 1 - protein, 118 aa, synthetic construct
QVQLQESGGGLVQAGGSLRLSLSASRYIFTLMGMRWYRRAPGKERELVASIQVGSDTNYRDSVKGRFTLSRDNAKNTVYLQMNSLKSDDTAVYYAAAPAYARRLHRYWGQGTQVTVSS

SEQ ID NO: 2 - protein, 122 aa, synthetic construct
QVQLQESGGGLVQAGGSLRLSLSASRYIFFWPGMGWFRRAPGKERELVASIPTGGATYYRDSVKGRFTLSRDNAKNTVYLQMNSLKSDDTAVYYAAAPPRPPATDKPLLPYWGQGTQVTVSS

SEQ ID NO: 3 - protein, 121 aa, synthetic construct
QVQLQESGGGLVQAGGSLRLSLSASRYIFLLQKMGWYRQAPGKERELVAGIHKGSDTNYGDSVKGRFTLSRDNAKNTVYLQMNSLKSDDTAVYYAAEPVPPRPRRRPPLPYWGQGTQVTVS

SEQ ID NO: 4 - protein, 126 aa, synthetic construct
QVQLQESGGGLVQAGGSLRLSLSASRYISFLKLMGWFRRAPGKERELVAGIHNGTNTNYRDSVKGRFTLSRDNAKNTVYLQMNSLKSDDTAVYYAAEPQPLWTITVTRREHPYDYWGQGTQVTVSS

SEQ ID NO: 5 - protein, 122 aa, synthetic construct
QVQLQESGGGLVQAGGSLRLSPSASWYISRWWGMGWFRRAPGKERELVASIDPGGDTNYPDSVKGRFTLSRDNAKNTVYLQMNSLKSDDTAVYYAAAAHPKLPTSPGKFQYWGQGTQVTVSS

SEQ ID NO: 6 - protein, 122 aa, synthetic construct
QVQLQESGGGLVQAGGSLRLSLSASRYIFLLPLMGWFRRAPGKERELVAGIARGTTTYYPDSVKGRFTLSRDNAKNTVYLQMNSLKSDDTAVYYAAAHRVTKPQATKPFRYWGQGTQVTVSS

SEQ ID NO: 7 - protein, 118 aa, synthetic construct
QVQLQESGGGLVQAGGSLRLSLSASRNIFWWPLMGWYRRAPGKEREFVASINSGTNTYYRDSVKGRFTLSRDNAKNTVYLQMNSLKSDDTAVYYAAAPFPAPETHTYWGQGTQVTVSS

SEQ ID NO: 8 - protein, 122 aa, synthetic construct
QVRLQESGGGLVQAGGSLRLSLSASGNIFRWWKMGWYRRAPGKERELVASIQTGANTYYADSVKGRFTLSRDNAKNTVYLQMNSLKSDDTAVYYAAEWLPSSQKNPYPHPYWGQGTQVTVSS

SEQ ID NO: 9 - protein, 125 aa, synthetic construct
QVQLQESGGGLVQAGGSLRLSLSASGSIFQWWAMRWFRRAPGKERELVASIHRGTVTNYPDSVKGRFTLSRDNAKNTVYLQMNSLKSDDTAVYYAAASTPDLSQNLLTDGLHRYWGQGTQVTVSS

SEQ ID NO: 10 - protein, 118 aa, synthetic construct
QVQLQESGGGLVQAGGSLRLSLSASRNIFLWLPMGWFRRAPGKEREFVASIGTGAATNYGDSVKGRFTLSRDNAKNTVYLQMNSLKSDDTAVYYAAARLAPHHRYSYWGQGTQVTVSS

SEQ ID NO: 11 - protein, 118 aa, synthetic construct
QVQLQESGGGLVQAGGSLRLSLSASRYIFPYLFMGWFRRAPGKERELVASIAAGTDTNYRDSVKGRFTLSRDNAKNTVYLQMNSLKSDDTAVYYAAARRPPKQTYLYWGQGTQVTVSS

SEQ ID NO: 12 - protein, 122 aa, synthetic construct
QVQLQESGGGLVQAGGCLRLSLSASRYIFHWCPMRWYRRAPGKERELVASIPRGGATYYRDSVKGRFTLSRDNAKNTVYLQMNSLKSDDTAVYYAAAHQAHPSPTATRYKYWGQGTQVTVSS

SEQ ID NO: 13 - protein, 126 aa, synthetic construct
QVQLQESGGGLVQAGGSLRLSLSASRYIFPWIVMGWFRRAPGKEREFVASINSGSTTNYRDSVKGRFTLSRDNAKNTVYLQMNSLKSDDTAVYYAAAQKEGRTTARTQINTYYGYWGQGTQVTVSS

SEQ ID NO: 14 - protein, 129 aa, synthetic construct
QVQLQESGGGLVQAGGSLRLSLSASRYIFAPRWMRWFRRAPGKERELVAGIQFGGDTNYGDSVKGRFTLSRDNAKNTVYLQMNSLKSDDTAVYYAAETLYYPRANPRPLPYWGQGTQVTVSSSEQIDNO

SEQ ID NO: 15 - protein, 126 aa, synthetic construct
QVQLQESGGGLVQAGGSLRLSLSASRYIFLYPLMGWFRRAPGKEREFVASIRRGTVTNYPDSVKGRFTLSRDNAKNTVYLQMNSLKSDDTAVYYAAATGNDRCCNRLGKQMFHPYWGQGTQVTVSS

SEQ ID NO: 16 - protein, 126 aa, synthetic construct
QVQLQESGGGLVQAGGSLRLSLSASRYIFVWAPMGWYRRAPGKERELVASIKAGTNTYYRDSVKGRFTLSRDNAKNTVYLQMNSLKSDDTAVYYAAAAPPDETRTVLTITNDHNYWGQGTQVTVSS

SEQ ID NO: 17 - protein, 122 aa, synthetic construct; identical to SEQ ID NO: 2

SEQ ID NO: 18 - protein, 118 aa, synthetic construct
QVQLQESGGGLVQAGGSLRLSLSASRYISWLQGMRWFRRAPGKEREFVASIIPGTNTNYGDSVKGRFTLSRDNAKNTVYLQMNSLKSDDTAVYYAAERRGQPLLHPYWGQGTQVTVSS

SEQ ID NO: 19 - protein, 118 aa, synthetic construct
QVQLQESGGGLVQAGGSLRLSLSASRYIFLPLFMGWYRRAPGKERELVAGINRGTTTNYPDSVKGRFTLSRDNAKNTVYLQMNSLKSDDTAVYYAAAAPSQTQYHKYWGQGTQVTVSS

SEQ ID NO: 20 - protein, 118 aa, synthetic construct
QVQLQESGGGLVQAGGSLRLSLSASRNIFSWMIMRWYRRAPGKERELVAGIKSGTDTNYRDSVKGRFTLSRDNAKNTVYLQMNSLKSDDTAVYYAAATPTQPTPHGYWGQGTQVTVSS

SEQ ID NO: 21 - protein, 118 aa, synthetic construct
QVQLQESGGGLVQAGGSLRLSLSASRTIFYWLGMRWFRRAPGKERELVAGIRTGATTNYRDSVKGRFTLSRDNAKNTVYLQMNSLKSDDTAVYYAAAPSVPPAYHPYWGQGTQVTVSS

SEQ ID NO: 22 - protein, 118 aa, synthetic construct
QVQLQESGGGLVQAGGSLRLSLSASRYIFVLPWMRWYRRAPGKERELVASITDGATTNYPDSVKGRFTLSRDNAKNTVYLQMNSLKSDDTAVYYAAEVNRPRQAYGYWGQGTQVTVSS

SEQ ID NO: 23 - protein, 122 aa, synthetic construct
QVQLQESGGGLVQAGGSLRLSLSASRSIFRCRYMRWFRQAPGKERELVASIICGTNTNYRDSVKGRFTLSRDNAKNTVYLQMNSLKSDDTAVYYAAETTEAHLRPVTNLMYWGQGTQVTVSS

SEQ ID NO: 24 - protein, 118 aa, synthetic construct
QVQLQESGGGLVQAGGSLRLSLSASRYIFWGPFMGWFRRAPGKERELVAGIAHGSNTNYADSVKGRFTLSRDNAKNTVYLQMNSLKSDDTAVYYAAAQAPVHPHHPYWGQGTQVTVSS

SEQ ID NO: 25 - protein, 118 aa, synthetic construct
QVQLQESGGGLVQAGGSLRLSLSASRYIFAWYRMGWFRRAPGKERELVASIQHGTITNYADSVKGRFTLSRDNAKNTVYLQMNSLKSDDTAVYYAAAQLGAPRQYRYWGQGTQVTVSS

SEQ ID NO: 26 - protein, 117 aa, synthetic construct
QVQLQESGGGLVQAGGSLRSLSASWNIFHFEWMRWFRRAPGKEREFVASIHRGSNTNYPDSVKGRFTLSRDNAKNTVYLQMNSLKSDDTAVYYAAAKSPFTVPLTYWGQGTQVTVSS

SEQ ID NO: 27 - protein, 118 aa, synthetic construct
QVQLQESGGGLVQAGGSLRLSLSASRSISWRGWMGWFRRAPGKEREFVASIGPGTNTNYADSVKGRFTLSRDNAKNTVYLQMNSLKSDDTAVYYAAEHLIPYYRFSYWGQGTQVTVSS

SEQ ID NO: 28 - protein, 127 aa, synthetic construct
QVQLQESGGGLVQAGGSLRLSLSASRSIFWWLGGMGWYRQAPGKERELVAGIAKGGNTNYRDSVKGRFTLSRDNAKNTVYLQMNSLKSDDTAVYYAAAQRGKSMRPLPTRRLSHSYWGQGTQVTVSS